(19) United States
(12) Patent Application Publication
(10) Pub. No.: US 2013/0149386 A1
(43) Pub. Date: Jun. 13, 2013

(54) CD36 AS BIOMARKER FOR STEATOSIS

(71) Applicant: REGION NORDJYLLAND, Aalborg Ost (DE)

(72) Inventor: Aase Handberg, Risskov (DK)

(73) Assignee: REGION NORDJYLLAND, Aalborg Ost (DE)

(21) Appl. No.: 13/707,883

(22) Filed: Dec. 7, 2012

Related U.S. Application Data
(60) Provisional application No. 61/567,723, filed on Dec. 7, 2011.

(51) Int. Cl. G01N 33/68

(52) U.S. Cl. CPC G01N 33/6893
USPC 424/520; 435/7.92; 514/449; 514/326; 514/342; 514/458; 514/210.02; 514/635; 514/182

(57) ABSTRACT

A method is provided for early treatment of steatosis, which method is based on early detection of steatosis based on the detection of CD36. CD36 is determined in a body fluid, and CD36 levels above a predetermined value is indicative of steatosis.

### REFERENCE TO RELATED APPLICATION

This application claims priority from U.S. Provisional Patent Application Ser. No. 61/567,723, filed Dec. 7, 2011, the entire content of which is incorporated by reference.

### FIELD OF THE INVENTION

The present invention relates to determination and monitoring of steatosis using measurement of the concentration of CD36 in a body fluid.

### BACKGROUND OF THE INVENTION

CD36 is a transmembrane glycoprotein expressed in a variety of tissues involved in tissue specific functions like angiogenesis, inflammation, lipid metabolism, atherosclerosis, and platelet activation (Collot-Teixeira G. Martin J. Dermott-Roe C. Poston R, McGregor J L. CD36 and macrophages in atherosclerosis. Cardiovasc Res 2007; 75(3): 468-77; Podrez E A, Byzova T V, Febbraio M, Salomon R G, Ma Y, Valiyaveettil M et al. Platelet CD36 links hyperlipidemia, oxidant stress and a prothrombotic phenotype. Nat Med 2007; 13(9): 1086-95; Silverstein R L. Inflammation, atherosclerosis, and arterial thrombosis: role of the scavenger receptor CD36. Cleve Clin J Med 2009; 76 Suppl 2: S27-S30). CD36 on the surface of muscle, liver, and fat tissue is involved in uptake of fatty acids, and elevated CD36 expression in these tissues and insulin resistance (IR) seems to be interrelated. CD36 on the surface of macrophages may initiate foam cell formation eventually leading to atherosclerotic lesions and thus may be an important risk factor of cardiovascular disease (CVD). The process of foam cell formation is initiated and enhanced by the binding of oxidized low density lipoprotein (oxLDL) to CD36 receptors with subsequent accumulation of cholesterol in the macrophage (Collot-Teixeira et al, 2007; Podrez et al, 2007; Silverstein et al. 2009). CD36 expression in macrophages appears to be induced by ox-LDL and IR, and plasma CD36 (sCD36) levels are increased in diabetic and insulin resistant patients, tightly correlated with IR and other cardiometabolic risk factors (Handberg A, Levin K, Hojlund K, Beck-Nielsen H. Identification of the oxidized low-density lipoprotein scavenger receptor CD36 in plasma: a novel marker of insulin resistance. Circulation 2006; 114(11): 1169-76). sCD36 also predicts the occurrence of diabetes in a 10-year follow-up of subjects initially non diabetic, not independently of fasting glucose and insulin.

It is difficult to diagnose steatosis and steato-hepatitis, and in fact, many individuals are asymptomatic, or may have secondary symptoms such as fatigue and right upper abdominal fullness or pain. Fatty liver is usually discovered incidentally because of abnormal liver function tests or hepatomegaly noted in unrelated medical conditions. One indication of fatty liver is elevated levels of alanine transaminase (ALT) and aspartate transaminase (AST). However, patients with nonalcoholic fatty liver disease may have normal transaminase levels. The ratio of AST/ALT is usually is less than 1 (in alcoholic liver disease, this ratio typically will be greater than 2) but may increase as the severity of the liver damage increases. Alkaline phosphatase may be elevated up to twice the upper limit of normal and γ-glutamyltransferase (GGT) also may be elevated. Imaging studies are also used in evaluation of fatty liver. Ultrasonography reveals a “bright” liver with increased echogenicity. Medical imaging can aid in diagnosis of fatty liver; fatty livers have lower density than spleen on computed tomography (CT) and fat appears bright in T1-weighted magnetic resonance images (MRIs). No medical imagery, however, is able to distinguish simple steatosis from advanced non-alcoholic steato-hepatitis (NASH). Histological diagnosis by liver biopsy is also applied when assessment of severity is indicated. Liver biopsy is the only reliable method of diagnosing NASH and determining the prognosis. Patients who are likely to have more advanced liver disease are therefore normally subject to biopsy. However, the role of liver biopsy in nonalcoholic fatty liver disease is controversial. Arguments against routine liver biopsy include the generally benign course of the disease in most cases, lack of established effective therapies, and risks of biopsy. Although liver biopsy generally is safe, transient pain occurs in many patients and severe pain in a smaller number of patients, and for a few patients, significant complications are seen. Biopsies is also associated with a, albeit relatively low, risk of death, which has been estimated to 0.03 percent.

### SUMMARY OF THE INVENTION

It has been found that increased concentration of CD36 in a body fluid sample from an individual correlates with an increased risk of steatosis or steato-hepatitis. Thus, the present invention relates to methods for diagnosing, classifying and monitoring steatosis or steato-hepatitis; i.e. methods for determining steato-hepatitis or increased likelihood of having or developing steatosis or steato-hepatitis. Additionally, circulating non-cell bound CD36 protein (soluble CD36, (sCD36)) or a fraction or fragment thereof optionally as part of a lipoprotein complex is provided as a useful diagnostic or predictive marker for steatosis or steato-hepatitis, in particular non-alcoholic steatosis or steato-hepatitis. The present inventors have found that the concentration of circulating CD36 is increased in patients with liver steatosis. Accordingly, increase in plasma CD36 concentration may be used as a biochemical marker of liver steatosis and progression thereof.

Thus, one aspect of the present invention relates to a method for diagnosing steatosis or steato-hepatitis in an individual, said method comprising in a sample from said individual

i) determining the concentration of a CD36 polypeptide or part thereof and/or
ii) correlating said concentration determined in i) to a standard level, and
iii) based on said correlation diagnosing said steatosis or steato-hepatitis.

The method of the present invention allows the diagnosis of steatosis or steato-hepatitis. However, the method can also be used for monitoring the progression of steatosis or steato-hepatitis in an individual, and/or for evaluating the risk of an individual acquiring steatosis or steato-hepatitis or developing more severe steatosis or steato-hepatitis. The method can also be used for evaluating the effect of a treatment provided to an individual, which has been diagnosed and subsequently treated for steatosis or steato-hepatitis.

Thus, other aspects of the present invention relates to methods of monitoring the progression of steatosis or steato-hepatitis in an individual, evaluating the risk of an individual acquiring steatosis or steato-hepatitis or developing more severe steatosis or steato-hepatitis and/or evaluating the effect of a treatment for steatosis or steato-hepatitis, where the methods comprise detecting CD36, for example by

i) determining the concentration of a CD36 polypeptide or part thereof and/or
ii) correlating said concentration determined in i) to a standard level, and
iii) based on said correlation monitoring the progression, evaluating the risk, and/or evaluating the effect of the treatment of steatosis or steato-hepatitis.

Another aspect relates to a method for determining steatosis or steato-hepatitis or increased likelihood of having or developing steatosis or steato-hepatitis in an individual, said method comprising

i. obtaining a body fluid sample from said individual
ii. contacting the body fluid sample with an anti-CD36 antibody
iii. determining the concentration of a CD36 polypeptide or part thereof in the body fluid
iv. correlating the concentration of CD36 polypeptide or part thereof to a predetermined standard level, and
v. determining that the individual has steatosis or steato-hepatitis or an increased likelihood of developing steatosis or steato-hepatitis based on a concentration of CD36 polypeptide or part thereof above the predetermined standard level.

The sample employed in the methods of the invention is preferably a body fluid sample, such as a plasma sample, for example a cell-free plasma sample. Accordingly, the level of circulating CD36 is determined, i.e. CD36 is preferably a soluble form of CD36 (sCD36). The methods of the invention is applicable to any individual, for example a human subject, and in a preferred embodiment, the individual has been diagnosed with hepatitis, chronic hepatitis, such as hepatitis C(CHC), for example genotype 1 CHC.

In the diagnosis of steatosis or steato-hepatitis, a level of CD36 above the average level in a population of human beings is indicative of steatosis or steato-hepatitis, and in a more specific embodiment, a level of soluble CD36 above the sCD36 level of the third quartile of a population of human beings is indicative of steatosis or steato-hepatitis. In another embodiment, an increase of CD36 concentration of at least 1.15 of the standard level is an indication of steatosis or steato-hepatitis.

The diagnostic/monitoring/evaluation methods are applicable to any form of steatosis or steato-hepatitis, but in a preferred embodiment, the assessed steatosis or steato-hepatitis is non-alcoholic steatosis or steato-hepatitis, for example induced by viral infection.

CD36 is preferably identified by

i) an amino acid sequence with SEQ ID NO: 4,
ii) an amino acid sequence having at least 90% sequence identity with a sequence of (i), or with a fragment thereof, and/or
iii) an amino acid sequence complementary to any of the sequences of (i) or (ii).

The methods may for example be performed by determining the CD36 polypeptide concentration by

i) providing a sample to be investigated,
ii) providing an anti-CD36 antibody,
iii) exposing the sample to the anti-CD36 antibody,
iv) optionally, removing excess unbound antibody,
v) optionally, exposing said CD36-antibody complex to at least a further antibody directed against said CD36-antibody complex, and
vi) detecting and quantifying the amount of said antibody

In one embodiment, the anti-CD36 antibody is bound to a solid phase, for example a dipstick, lateral flow test, or a microtitre plate.

Thus, in one preferred embodiment, CD36 is detected by an immunological method, for example a solid phase ELISA enzyme immunoassay, wherein the sample is contacted with the anti-CD36 antibody bound to a solid phase, such as a microtiter plate.

The anti-CD36 antibody can be any detectable and specific antibody, both monoclonal and polyclonal CD36-specific antibodies, and/or fragments thereof. Several anti-CD36 antibodies are available to the skilled person. Anti-CD36 antibodies are also commercially available. For example, the antibody used in the present invention is selected from the group of antibodies consisting of sc5522 (CD36 (N-15), goat IgG (epitope N-terminus (h)), sc9154 (CD36 (H-300), rabbit IgG (epitope 1-300 (h)), and sc7309 (CD36 (SMf), mouse IgM).

In another aspect the present invention relates to the use of a kit comprising at least one detection member. It is appreciated that the detection member is at least one antibody, wherein said antibody is directed against CD36 polypeptide or part thereof. Detection members of the kit are preferably anti-CD36 antibodies, such as described above.

Another aspect relates to a method of treatment treating steatosis or steato-hepatitis in an individual, said method comprising in a sample from said individual

i) determining the concentration of a CD36 polypeptide or part thereof
ii) correlating the concentration to a standard level, and
iii) selecting patients having an elevated level of CD36, and
iv) providing a treatment for steatosis or steato-hepatitis.

Any suitable treatment may be provided given the specific circumstances of the patient. The method allows early detection of steatosis at early stage, which allows treatment at an early stage, where the condition is reversible. However, in a preferred embodiment, the treatment is selected from the group consisting of weight loss (surgical or medical (e.g. using Orlistat, Rimonabant and/or a Glucagon-like protein-1-receptor agonist)), alcohol restriction, diet changes (preferably towards low-carbohydrate diets), physical exercise, improving metabolic risk factors, providing antiglycemic drug agents/Insulin-Sensitizing Medications such as Thiazolidinediones (e.g. Pioglitazone), Metformin, Antioxidant therapy (e.g. vitamin E), Cytoprotective agents (e.g. Ursodeoxycholic acid (UDCA)), 3-hydroxy-3-methylglutaryl-coenzyme, Ezetimibe, and/or Angiotensin-receptor blockers.

### BRIEF DESCRIPTION OF DRAWINGS

FIG. 1. Markers of insulin resistance, carotid atherosclerosis and fatty liver across quartiles of sCD36. Percentage of the RISC cohort (N=1296) with either IGT or IFG representing impaired glucose regulation (FIG. 1A), or presence of the metabolic syndrome (FIG. 1B), values of insulin sensitivity (M/I in (μmol.min-1.kgFFM-1.nmol/l-1) measured by the eugly-cemic-hyperglycemic clamp (FIG. 1C), carotid intima-media thickness in mm (FIG. 1D), fatty liver index (FIG. 1E), and liver fat percentage (FIG. 1F) across quartiles (Q1-Q4) of sCD36. Data are given as mean±SEM. Except for IGT/IFG, the P values are based on statistical analyses of log-normalized values. P<0.05 vs Q2, P<0.05 vs Q3, and P<0.05 vs. Q4 in post-hoc analysis.

FIG. 2. Distribution of sCD36 in the study populations. Distribution of log sCD36 among all study participants, N=1296 (FIG. 2A), and in the log normal study population (N=1029) (FIG. 2B). The dashed line indicates the mean levels of log sCD36 (−0.38) at the lower end of the distribution at which frequencies starts to increase again.

FIG. 3. The level of steatosis associated with quartiles of sCD36 plasma levels.

### DETAILED DESCRIPTION OF THE INVENTION

### Definitions

The term “diagnosis” as used herein refers to methods by which the skilled person can estimate and/or determine whether or not a patient is suffering from a given disease or condition, such as steatosis or steato-hepatitis. The skilled person often makes a diagnosis on the basis of one or more diagnostic indicators, i.e., in the amount or concentration of a marker, or change in amount of marker which is indicative of the presence, severity, or absence of the condition.

The term “correlating” as used herein, refers to comparing the presence or amount of the marker(s) in a patient to its presence or amount in individuals suffering from or at risk of suffering from, a given condition; or in persons known to be free of a given condition. As discussed above, a marker level in a patient sample can be compared to a level known to be associated with a specific diagnosis. The sample's marker level is said to have been correlated with a diagnosis; that is, the skilled person can use the marker level to determine whether the patient suffers from a specific type diagnosis, and respond accordingly. Alternatively, the sample's marker level can be compared to a marker level known to be associated with a good outcome (e.g., the absence of disease, etc.).

It is appreciated that the term ‘marker’ in the present invention refers to CD36 polypeptide and/or CD36 encoding nucleic acid molecule.

The term “determining the diagnosis” as used herein refers to methods by which the skilled person can determine the presence or absence of a particular disease or condition, such as steanosis, in a patient. The term “diagnosis” does not refer to the ability to determine the presence or absence of a particular or condition disease with 100% accuracy. Instead, the skilled person will understand that the term “diagnosis” refers to an increased probability that a certain disease or condition is present in the subject. In preferred embodiments, a diagnosis indicates about a 5% increased chance that a disease or condition is present, about a 10% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−2%.

### Diagnosis, Classification, Evaluation and Monitoring Steatosis or Steato-Hepatitis

The present invention is based on the fact that level of circulating CD36, sCD36, is increased in patients with steatosis. Steatosis is also known as fatty liver or fatty liver disease (FLD), and is a reversible condition where large vacuoles of triglyceride fat accumulate in liver cells. Steatosis and steato-hepatitis is characterized by abnormal retention of lipids within liver cells.

The term “steatosis” as used herein, is meant to include all stages of fatty liver development from the initial asymptomatic phase with small amounts of accumulated fat in the liver to the later stages of more severe fatty liver. More severe later stages of the disorder are referred to as steato-hepatitis, which may be accompanied by a progressive inflammation of the liver, hepatocyte necrosis and/or cirrhosis, characterized by replacement of liver tissue by fibrosis, scar tissue and regenerative nodules (lumps that occur as a result of a process in which damaged tissue is regenerated), which gradually and ultimately leads to loss of liver function.

The disease stage of a fatty liver can be defined in terms of a “Fatty liver index”. The fatty liver index (FLI) uses an algorithm based on body mass index (BMI), waist circumference, triglycerides and GGT with an accuracy of 0.84 (95% confidence interval 0.81-0.87) in detecting fatty liver. In one embodiment of the present invention, a patient has steatosis or steato-hepatitis, or increased likelihood of having or developing steatosis or steato-hepatitis, when the FLI is above 20, such as above 30, such as above 40, such as above 50, such as above 60, such as above 70, such as above 80, such as above 90. Preferably, an FLI above 60 is indicative of steatosis or steato-hepatitis, or increased likelihood of having or developing steatosis or steato-hepatitis. Conversely, patients having an FLI below 20 are generally not having steatosis or steato-hepatitis, or have low likelihood of having or developing steatosis or steato-hepatitis.

Another method of evaluating steatosis or steato-hepatitis is by estimating the “Liver fat percentage” (LF %). Liver fat percentage (LF %) uses an algorithm based on presence of MetSy and T2DM as well as fasting insulin, aspartate- and alanine-aminotransferases to predict the degree of liver fat accumulation.

The present invention, however, provides a simple and reliable methodology for determining steatosis or steato-hepatitis or the risk thereof, based on the detection of circulating CD36.

Steatosis can arise from several different causes. One of the most prominent causes of steatosis is excessive alcohol intake. steatosis arising as a result of excessive alcohol intake, usually more than 20-30 g alcohol or more than 2 standard drinks per day, is also referred to as alcohol-dependent steatosis or steato-hepatitis or alcoholic steatosis or steato-hepatitis. Other causes of steatosis are collectively called non-alcoholic steato-hepatitis (NASH). One major cause of steatosis is obesity (with or without effects of insulin resistance). Obese individuals are more prone to steatosis. However, the condition is also associated with other diseases that influence fat metabolism. Alcoholic steatosis and non-alcoholic steatosis are not easily distinguished morphologically, and both show micro-vesicular and macrovesicular fatty changes at different stages.

Another major cause of steatosis or steato-hepatitis is viral infection. Viral hepatitis may exist in acute (recent infection, relatively rapid onset) or chronic forms. The most common causes of viral hepatitis are the five unrelated hepatotropic viruses Hepatitis A, Hepatitis B, Hepatitis C, Hepatitis D, and Hepatitis E. In addition to the hepatitis viruses, other viruses that can also cause hepatitis include Herpes simplex, Cytomegalovirus, Epstein-Barr virus, and Yellow fever.

Hepatitis C is caused by hepatitis C virus (HCV). The hepatitis C virus is usually detectable in the blood by PCR within one to three weeks after infection, and antibodies to the virus are generally detectable within three to 15 weeks. Spontaneous viral clearance rates are highly variable; between 10 and 60% of persons infected with HCV clear the virus from their bodies during the acute phase, measured by normalization of the liver enzymes alanine transaminase (ALT) and aspartate transaminase (AST), and plasma HCV-RNA clearance. However, persistent infections are common and most patients develop chronic hepatitis C, where the infection lasts more than 6 months.

There are six major genotypes of the hepatitis C virus, which are indicated numerically as genotype 1, 2, 3, 4, 5, and 6. The prevalence of HCV genotype varies geographically. Approximately 80% of hepatitis C patients in the United States have genotype 1, and genotype 4 is more common in the Middle East and Africa.

Metabolic factors may be able to interfere with the natural history of patients with genotype 1 chronic hepatitis C (G1 CHC). In particular, steatosis has been systematically associated with the severity of fibrosis, the occurrence of hepatocellular carcinoma (HCC) and with a lower likelihood to achieve sustained viral response (SVR) after standard antiviral therapy. The clinical relevance of steatosis in G1 CHC patients appears of further interest considering its high prevalence in this clinical setting, due to both an intrinsic ability of HCV to (in)directly promote liver fat accumulation, and to host metabolic factors, namely visceral obesity and insulin resistance (IR).

The present invention provides methods for diagnosing steatosis or steato-hepatitis on the basis of circulating CD36; thus the invention relates to methods for diagnosing steatosis or steato-hepatitis in an individual, said method comprising in a sample from said individual

i) determining the concentration of a CD36 polypeptide or part thereof and/or
ii) correlating said concentration determined in i) to a standard level, and
iii) based on said correlation diagnosing said steatosis or steato-hepatitis.

The invention also provides a method for determining steatosis or steato-hepatitis or increased likelihood of having or developing steatosis or steato-hepatitis in an individual, said method comprising

i. obtaining a body fluid sample from said individual
ii. contacting the body fluid sample with an anti-CD36 antibody
iii. determining the concentration of a CD36 polypeptide or part thereof in the body fluid
iv. correlating the concentration of CD36 polypeptide or part thereof to a predetermined standard level, and
v. determining that the individual has steatosis or steato-hepatitis or an increased likelihood of developing steatosis or steato-hepatitis based on a concentration of CD36 polypeptide or part thereof above the predetermined standard level.

Similar methods may also be used for monitoring the progression of steatosis or steato-hepatitis in an individual, and/or for evaluating the risk of an individual acquiring steatosis or steato-hepatitis or developing more severe steatosis or steato-hepatitis; and the same methods can also be used for evaluating the effect of a treatment provided to an individual, which has been diagnosed and subsequently treated for steatosis or steato-hepatitis. Thus, the invention also relates to methods of monitoring the progression of steatosis or steato-hepatitis in an individual, evaluating the risk of an individual acquiring steatosis or steato-hepatitis or developing more severe steatosis or steato-hepatitis and/or evaluating the effect of a treatment for steatosis or steato-hepatitis, where the methods comprise detecting CD36, for example by

i) determining the concentration of a CD36 polypeptide or part thereof and/or
ii) correlating said concentration determined in i) to a standard level, and
iii) based on said correlation monitoring the progression, evaluating the risk, and/or evaluating the effect of the treatment of steatosis or steato-hepatitis.

The provided methods can be applied to any type of steatosis or steato-hepatitis, including steatosis or steato-hepatitis of any underlying cause, and also to any and all stages of steatosis or steato-hepatitis. In one embodiment, the methods are applied to individuals having alcoholic steatosis or steato-hepatitis. However, in another embodiment, the methods are applied to individuals with non-alcoholic steatosis or steato-hepatitis. In one specific embodiment, the methods are applied to an individual having a viral infection, such as hepatitis. In a preferred embodiment, the individual has chronic hepatitis, in particular chronic hepatitis C, and in a most preferred embodiment, the individual has genotype 1 hepatitis C.

The concentration of CD36 polypeptide is determined in methods of the invention and correlated to a standard level. The outcome of the correlation can be compared to concentrations of CD36 polypeptide determined at other time points in said individual. By monitoring the concentration of CD36 polypeptide and/or CD36 encoding nucleic acid molecule in an individual at different time points the progress of the steatosis or steato-hepatitis can be followed. Such monitoring of an individual can be useful for determining a suitable treatment of an individual.

In a further aspect the invention relates to a method for diagnosing an individual at risk of having and/or acquiring a steatosis or steato-hepatitis, said method comprising in a sample from said individual, i) determining the concentration of a CD36 polypeptide or part thereof and/or ii) determining the concentration of a CD36 encoding nucleic acid molecule or part thereof, iii) correlating said concentration determined in i) and/or ii) to a standard level, and iv) based on said correlation according to iii) diagnosing whether said individual is at risk of having and/or acquiring a steatosis or steato-hepatitis.

### Method of Treatment of Steatosis or Steato-Hepatitis

One aspect of the present invention relates to a method of treating steatosis in an individual, wherein steatosis is determined by a method of the invention, as defined elsewhere herein, and providing a treatment of steatosis. This approach allows to intervene with an early treatment of steatosis, for example in the asymptomatic stage of the condition. In this method of treatment, the steatosis can be reversible, and steatosis can potentially be cured.

In a preferred embodiment, a method is provided of treating steatosis in an individual, said method comprising in a sample from said individual

i) obtaining a body fluid sample from said individual,
ii) contacting the body fluid sample with an anti-CD36 antibody,
iii) determining the concentration of a CD36 polypeptide or part thereof in the body fluid,
iv) correlating the concentration of CD36 polypeptide or part thereof to a predetermined standard level,
v) determining that the individual has steatosis or an increased likelihood of developing steatosis based on a concentration of CD36 polypeptide or part thereof above the predetermined standard level, and
iv) providing a treatment for steatosis.

Any suitable treatment may be provided given the specific circumstances of the patient. However, in a preferred embodiment, the treatment is selected from the group consisting of weight loss (surgical or medical (e.g. using Orlistat, Rimonabant and/or a Glucagon-like protein-1-receptor agonist)), alcohol restriction, diet changes (preferably towards low-carbohydrate diets), physical exercise, improving metabolic risk factors, providing antiglycemic drug agents/Insulin-Sensitizing Medications such as Thiazolidinediones (e.g. Pioglitazone), Metformin, Antioxidant therapy (e.g. vitamin E), Cytoprotective agents (e.g. Ursodeoxycholic acid (UDCA)), 3-hydroxy-3-methylglutaryl-coenzyme, Ezetimibe, and/or Angiotensin-receptor blockers.

### CD36

For the purposes of the present invention the term “CD36” used in phrases, such as “circulating CD36”, or “non-cell bound CD36”, and in the claims, includes CD36 protein or a fragment thereof which is recognised by a specific antibody against CD36, such as sc5522, sc9154, and sc7309. This includes full length CD36 protein, a polypeptide or peptide fragment thereof, as well as such protein or fragment(s) thereof present in a high molecular weight plasma fraction complex comprising lipoprotein thereof all of which are preferably soluble (sCD36). The terms circulating CD36 and soluble CD36 (sCD36) may be used as synonyms herein. The circulating CD36 may be secreted from caveolae or which is part of a microparticle comprising membrane portions originating from a caveola membrane which has been released from a cell membrane and is present in the blood circulation. Caveolae of interest in this connection may be present in the cell membrane of cells, such as hepatocytes, adipocytes, macrophages, monocytes, platelets and thrombocytes.

CD36 is a 471 amino acid, transmembrane protein (having 1 or 2 membrane spanning domains at amino acid residue positions 439-460 and possibly 7-28). CD36 is a highly glycosylated, 88 kDa glycoprotein with palmitoyl binding sites. CD36 is present in caveolae where it may play a role in the mediation of cellular cholesterol movement into and out of cells.

Molecular Families in which CD36 is a Member:
CD36-->SR-B class-->host defense scavenger receptors-->scavenger receptor superfamily

Molecular Structure of CD36:

471 amino acid residues;
Transmembrane region (residues 439-465) and,
438 amino acid amino-terminal region may be entirely extracellular or may have a second potential transmembrane region near the amino terminal end;
aa short cytoplasmic tail (residues 466-471);

Within the extracellular region resides a hydrophobic region which probably associates with the outer cell membrane (residues 184-204).

The molecular mass of CD36 is reported to be dependent on cell type as shown below:

Platelets 88 kDa/113 kDa

### Fetal Erythrocytes 78 kDa

Mammary epithelial Cells 85 kDa
Erythroleukeimic 88 kDa, 85 kDa, 57 kDa8

### HeLa 85 (160) kDa 85 kDa

Dermal Microvascular endothelial cells 80-90 kDa

In post-transcriptional modification of CD36 two alternate CD36 mRNA forms have been identified. The first mRNA type is expressed in HEL cells and omits amino acid residues 41-143. The second mRNA type has not yet been translated but in which the last 89 residues have been omitted. Thus, CD36 is also found in forms in which the open reading frame is 90-1708 and 357-1775, respectively.

### Post-Translational Modification of CD36:

CD36 is purported to be heavily glycosylated, with 10 N-linked glycosylation sites in the extracellular portion. Glycosylation has been suggested to confer its resistance to proteolytic cleavage;
Threonine 92 has been shown to be phosphorylated;
CD36 is also palmitoylated on both N- and C-terminal cytoplasmic tails.
Proteins and DNA Elements which Regulate Transcription of CD36 Molecule:
Oct-2: The first gene shown to be regulated by the Oct-2 transcription factor during B cell differentiation;
PEBP2: The PEBP2/CBF transcription factors may be important for the constitutive expression of CD36 in monocyte;
CBF: The PEBP2/CBF transcription factors may be important for the constitutive expression of CD36 in monocyte.

Substrates for CD36 are unknown. It may be possible that CD36 regulates autophosphorylation of residue Thr92. Enzymes which modify CD36 are unknown. It may be possible that Thr92 is phosphorylated by extracellular threonine kinase(s).

Intracellular signalling is probably associated with phosphorylation of Fyn, Lyn and Yes, but the manner by which the cytoplasmic tail interacts with these PTKs is unknown.

### Main Cellular Expression of CD36:

CD36 is expressed on platelets, mature monocytes and macrophages, microvascular endothelial cells, mammary endothelial cells, during stages of erythroid cell development and on some macrophage derived dendritic cells, muscle cells, liver cells, and adipocytes.

The physiological events regulated by CD36 ligation are still very much unknown. Up to 50% of oxidized LDL are ingested through CD36, thus CD36 appears to be a major scavenger receptor. However, given the apparent absence of disease states in CD36 deficient subjects, other mechanisms appear to be capable of compensating for its absence.

### Sequence Identity

Functional equivalents and variants are used interchangeably herein. In one preferred embodiment of the invention there is also provided variants of CD36 and variants of fragments thereof. When being polypeptides, variants are determined on the basis of their degree of identity or their homology with a predetermined amino acid sequence, said predetermined amino acid sequence being SEQ ID NO: 4, and when the variant is a fragment, a fragment of that amino acid sequence.

Accordingly, variants preferably have at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence.

The following terms are used to describe the sequence relationships between two or more polynucleotides: “predetermined sequence”, “comparison window”, “sequence identity”, “percentage of sequence identity”, and “substantial identity”.

A “predetermined sequence” is a defined sequence used as a basis for a sequence comparision; a predetermined sequence may be a subset of a larger sequence, for example, as a segment of a full-length DNA, transcriptional product thereof, gene sequence given in a sequence listing, such as a polynucleotide sequence of SEQ ID NO:1, SEQ ID NO: 2 or SEQ ID NO: 3 or may comprise a complete DNA or gene sequence. Generally, a predetermined sequence is at least 20 nucleotides in length, frequently at least 25 nucleotides in length, and often at least 50 nucleotides in length.

Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a predetermined sequence of at least 20 contiguous nucleotides and wherein the portion of the polynucleotide sequence in the comparison window may comprise additions or deletions (i.e., gaps) of 20 percent or less as compared to the predetermined sequence (which does not comprise additions or deletions) for optimal alignment of the two sequences.

Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a predetermined sequence over a comparison window of at least 20 nucleotide positions, frequently over a window of at least 25-50 nucleotides, wherein the percentage of sequence identity is calculated by comparing the predetermined sequence to the polynucleotide sequence which may include deletions or additions which total 20 percent or less of the predetermined sequence over the window of comparison. The predetermined sequence may be a subset of a larger sequence, for example, as a segment of the full-length SEQ ID NO:1, SEQ ID NO:2 and/or SEQ ID NO:3 polynucleotide sequence illustrated herein.

By the term “transcriptional or translational products” is meant herein products of gene transcription, such as a RNA transcript, for example an unspliced RNA transcript, a mRNA transcript and said mRNA transcript splicing products, and products of gene translation, such as polypeptide(s) translated from any of the gene mRNA transcripts and various products of post-translational processing of said polypeptides, such as the products of post-translational proteolytic processing of the polypeptide(s) or products of various post-translational modifications of said polypeptide(s).

As used herein, the term “transcriptional product of the gene” refers to a pre-messenger RNA molecule, pre-mRNA, that contains the same sequence information (albeit that U nucleotides replace T nucleotides) as the gene, or mature messenger RNA molecule, mRNA, which was produced due to splicing of the pre-mRNA, and is a template for translation of genetic information of the gene into a protein.

As used herein, the term “translational product of the gene” refers to a protein, which is encoded by the CD36 gene.

The invention specifically relates to methods involving the step of determining the concentration of a CD36 polypeptide or part thereof in
(1) variant proteins corresponding to the protein identified as SEQ ID NO: 4 or variants, or fragments thereof,
(ii) polypeptide sequences having at least 90% identity with the variant proteins, or fragments thereof, of (i),

Thus, it is an object or embodiment of the invention to use the above identified variant proteins for the purpose of

i) diagnosing steatosis or steato-hepatitis in an individual
ii) classifying steatosis or steato-hepatitis in an individual
iii) monitoring the progression steatosis or steato-hepatitis in an individual
iv) evaluating the risk of an individual acquiring steatosis or steato-hepatitis or developing more severe steatosis or steato-hepatitis
v) evaluating the effect of a treatment provided to an individual, which has been diagnosed and subsequently treated for steatosis or steato-hepatitis.

Sequence identity is determined in one embodiment by utilising fragments of CD36 peptides comprising at least 25 contiguous amino acids and having an amino acid sequence which is at least 80%, such as 85%, for example 90%, such as 95%, for example 99% identical to the amino acid sequence of SEQ ID NO: 4, wherein the percent identity is determined with the algorithm GAP, BESTFIT, or FASTA in the Wisconsin Genetics Software Package Release 7.0, using default gap weights.

### Conservative Amino Acid Substitutions:

Substitutions within the groups of amino acids, shown below, are considered conservative amino acid substitutions. Substitutions between the different groups of amino acids are considered non-conservative amino acid substitutions.

P, A, G, S, T (neutral, weakly hydrophobic)
Q, N, E, D, B, Z (hydrophilic, acid amine)
H, K, R (hydrophilic, basic)
F, Y, W (hydrophobic, aromatic)
L, I, V, M (hydrophobic)
C (cross-link forming)

### Individuals

The present invention relates to individuals, for example mammals and in particular humans of any sex or age. The individuals may be asymptomatic with regard to steatosis or steato-hepatitis. Thus, the individual of the present invention may never have suffered from steatosis or steato-hepatitis or any associated diseases. However, it is also within the scope of the present invention that the individual of the present invention may previously have been suffering from steatosis or steato-hepatitis or related diseases. The individual may also have one or more symptoms of steatosis or steato-hepatitis as described herein above.

In one embodiment, an individual of the present invention has a viral infection, such as hepatitis. In a preferred embodiment, the individual has chronic hepatitis, in particular chronic hepatitis C, and in a most preferred embodiment, the individual has genotype 1 hepatitis C.

### Sample

The sample according to the invention may be a body fluid sample. However, in a preferred embodiment, the sample is a blood sample, in particular a cell-free plasma or serum sample. That the plasma is cell-free may be verified microscopically or by flow cytometry following centrifugation of the blood sample. Centrifugation causes the blood cells to be separated from the plasma. Centrifugation of a blood sample in the range of 1,500 to 3000 G for 5-20 minutes, for example 1850 G for 10 minutes, will result in a cell-free plasma. In one embodiment the sample is a peripheral venous blood sample. A preferred embodiment is a cell-free sample, preferably a cell-free plasma sample. In another embodiment, the sample is cleared of microparticles. This is obtained for example by filtration using a filter with a cut-off value (pore size) of 0.1-0.5 μm. Alternatively, the sample, for example the cell free sample, can be centrifuged 10.000-20.000, such as ˜16.000 G, for 30-45 min. In this case the supernatant will be free of microparticles.

Suitable plasma preparations may be plasma from heparin-stabilised blood, Citrate-stabilised or EDTA-stabilised blood.

The samples may be fresh or frozen. For example the samples are frozen at −80° C. and are thawed only once prior to determining the concentration of CD36. In a preferred embodiment, the sample is frozen and thawed at least once, but more preferred twice or three times, prior to determining the concentration of CD36. Cycles of freezing and thawing help disrupting high-molecular complexes in the sample, wherein CD36 may be bound. Freezing and thawing, therefore in one embodiment promote the exposures of CD36 polypeptide or part thereof for antibody binding, which allows more precise CD36 quantification. The procedure for disruption of high-molecular complexes should be standardized in the clinical use of the analysis, where predetermined values are used for determining steatosis or steato-hepatitis. In one embodiment, the sample is frozen and thawed at least once, such as 1-6 times, such as 3-4 times.

### Detection Method

The methods of the present invention comprise determining concentration of a CD36 polypeptide or part thereof in a sample from an individual, and correlating that CD36 concentration determined to a standard level. Based on the correlation between the determined and predetermined CD36 concentration, steatosis or steato-hepatitis can be diagnosed in the tested individual. The level of CD36 may be determined by any method available to the skilled person. In a preferred embodiment, CD36 is determined in a blood sample, such as a cell free plasma sample. A number of convenient immunological methods exists for determining polypeptides in a blood sample, and thus, in a preferred embodiment, CD36 is determined by immunological methods.

### Immunological Methods

In the context of the present invention, “immunological methods” are understood as meaning analytical methods based on immunochemistry, in particular on an antigen-antibody reaction. Examples of immunological methods include immunoassays such as radioimmunoassay (RIA), enzyme immunoassay (EIA, combined with solid-phase technique: ELISA (Enzyme Linked Immuno Sorbent Assay)) or else immunofluorescence assays. The immunoassay is carried out by exposing the sample to be investigated to a CD36-binding antibody and detecting and quantifying the amount of CD36 bound to this antibody. In these assays, detection and quantification is carried out directly or indirectly in a known manner. Thus, detection and quantification of the antigen-antibody complexes is made possible by using suitable labels which may be carried by the antibody directed against CD36 and/or by a secondary antibody directed against the primary antibody. Depending on the type of the abovementioned immunoassays, the labels are, for example, radioactive labels, chemiluminescent labels, fluorescent dyes or else enzymes, such as phosphatase or peroxidase, which can be detected and quantified with the aid of a suitable substrate.

In one embodiment of the invention, the immunological method is carried out with the aid of a suitable solid phase. Suitable solid phases which may be mentioned include the microtiter plates made of polystyrene or membranes (for example made of polyvinylidene difluoride, PVDF) which are adapted and suitable for the ELISA technique.

To carry out a method according to the invention, a suitable sample, such as a liquid patient sample (e.g. a blood sample, such as a cell free plasma sample), is applied to the solid phase. The sample is preferably a plasma sample wherein the CD36 or fraction thereof is present in unbound form or is present in a form which could be bound to its ligand LDL. CD36 can also be associated with microparticles. sCD36 is present in complex with a high molecular fraction in plasma, and therefore it is preferred to disrupt high molecular complexes (e.g. lipid-protein complexes) in the sample are prior to the exposure of the sample to anti-CD36 antibody. One preferred embodiment comprises freezing and thawing the samples to be tested, such that high molecular complexes (lipid-protein complexes) are disrupted and/or degraded. A standardized procedure for disrupting the high-molecular complexes should be used. In a preferred embodiment of the present invention, the sample is frozen and thawed at least once, such as 1-6 times, such as 3-4 times. The sample is frozen below 0°, for example frozen to −20°. In one embodiment, the sample is first frozen to −85° C. to −70° C. (e.g. ˜−80° C.), once, and then at least once to −10° C. to −38° C. (e.g. −15° C. to −25° C., such as ˜−20° C.), or more preferably 2-5 times, such as 3-4 times. The sample may be frozen for a few minutes to several hours or days. The important issue is that the sample is frozen, which serves to disrupt high molecular complexes. Thus, the incubation time for freezing is not of vital importance. However, the handling of the samples should be standardized as far as possible.

In one aspect the invention relates to a method, wherein the CD36 polypeptide concentration is determined by i) providing a sample to be investigated, ii) providing an anti-CD36 antibody, iii) exposing the sample to the anti-CD36 antibody, iv) optionally, exposing said CD36-antibody complex to at least a further antibody directed against said CD36-antibody complex, and v) detecting and quantifying the amount of said anti-CD36 antibody or said further antibody.

In a more specific embodiment of the methods of the invention, the concentration of human CD36 polypeptide or part thereof is determined in a plasma sample by a solid phase ELISA enzyme immunoassay which comprises the steps of

(i) providing a plasma sample to be investigated,
(ii) providing an anti-CD36 antibody as defined herein,
(iii) exposing the sample to be investigated to a solid phase and the antibody, and
(iv) detecting and quantifying the amount of the antibody which binds to CD36; and
in a more preferred embodiment of the invention there is provided a method for determining the concentration human CD36 polypeptide or part thereof in a plasma sample by solid phase ELISA enzyme immunoassay which comprises the steps of
(i) providing a plasma sample to be investigated,
(ii) providing an anti-CD36 antibody,
(iii) exposing the sample to be investigated to the anti-CD36 antibody bound to a solid phase,
(iv) optionally exposing the CD36-antibody complex to a second anti-CD36 antibody, and
(iv) detecting and quantifying the amount of the antibody which binds to CD36,
and wherein the solid phase preferably is a microtiter plate, the CD36 is preferably present (part of) in a high molecular weight plasma fraction said high molecular weight plasma fraction preferably being a CD36-lipoprotein complex; said anti-CD36 antibody is preferably selected from the group consisting of monoclonal antibodies as specified below; said determination is preferably carried out by a solid phase enzyme immunoassay, wherein, in the enzyme immunoassay, the sample is exposed to a first, human CD36-binding antibody, and the amount of bound CD36 is measured using a second CD36-binding antibody carrying an enzyme label, where the measurement is carried out by an enzyme-catalyzed colour reaction or chemiluminescence.

It is also within the scope of the present invention to determine the concentration of CD36 by multiplex suspension array technique.

### Antibodies

Methods of the present invention employ an antibody directed to an epitope of CD36 polypeptide or part thereof as described elsewhere herein. Such an antibody may be used in methods of the present invention for determining steatosis or steato-hepatitis or increased likelihood of having or developing steatosis or steato-hepatitis in an individual, for the diagnosis of steatosis or steato-hepatitis, monitoring the progression of steatosis or steato-hepatitis in an individual, evaluating the risk of an individual acquiring steatosis or steato-hepatitis or developing more severe steatosis or steato-hepatitis, or evaluating the effect of a treatment provided to an individual, which has been diagnosed and subsequently treated for steatosis or steato-hepatitis. Thus, epitope in this context covers any epitope capable of being recognized by an antibody or a binding fragment thereof.

The term “antibody” as used herein includes both polyclonal and monoclonal antibodies, as well as fragments thereof, such as, Fv, Fab and F(ab)2 fragments that are capable of binding antigen or hapten. It includes conventional murine monoclonal antibodies as well as human antibodies, and humanized forms of non-human antibodies, and it also includes ‘antibodies’ isolated from phage antibody libraries.

The antibodies of the present invention may be polyclonal or monoclonal and may be produced by in vivo or in vitro methods known in the art. Thus, the anti-CD36 antibody is selected from the group consisting of monoclonal and polyclonal CD36-specific antibodies.

A monoclonal antibody is an antibody produced by a hybridoma cell. Methods of making monoclonal antibody-synthesizing hybridoma cells are well known to those skilled in the art, e.g, by the fusion of an antibody producing B lymphocyte with an immortalized B-lymphocyte cell line.

A polyclonal antibody is a mixture of antibody molecules (specific for a given antigen) that has been purified from an immunized (to that given antigen) animal's blood, where a non-limiting example is antibody molecules from rabbit. Such antibodies are polyclonal in that they are the products of many different populations of antibody-producing cells.

The invention also pertains to mixtures of monoclonal and/or polyclonal antibodies. Also a mixture of at least two monoclonal antibodies is within the scope of the present invention. It is appreciated that the mixture may comprise 3, 4, 5, 6, 7, 8, 9, 10, or 15 monoclonal antibodies.

The anti-CD36 antibody used herein may be a monoclonal antibody as described above, and/or an isolated polyclonal antibody, obtainable by an immunization process in which purified or recombinant human CD36 is used as antigen component and the antibody is preferably specific for various fractions of the CD36 protein which is present in human blood plasma in soluble form, optionally as a part of a CD36-lipoprotein complex, such as CD36 or a fraction thereof bound to Low Density Lipoprotein (LDL), IDL (Intermediate Density Lipoprotein or VLDL (Very Low Density Lipoprotein) which may be present in a high molecular weight fraction of cell-free plasma. In one embodiment the CD36-lipoprotein complex comprises oxidized forms of lipoproteins. For example, sCD36 is associated with oxidized LDL (oxLDL), oxidized LDL, oxidized IDL and/or oxidized VLDL. In yet another embodiment sCD36 is found in complex with oxidized HDL.

Any specific antibody directed against CD36 may be used according to the present invention. In one embodiment the antibody is selected from the group consisting of sc7309 (CD36 (SMf), mouse IgM), and polyclonal CD36 specific antibodies, such as sc5522 (CD36 (N-15), goat IgG, epitope N-terminus (h)), sc9154 (CD36(H-300), rabbit IgG, epitope 1-300 (h)), such as sc5522 and sc9154 (both from Santa Cruz Biotechnology Inc., Santa Cruz, Calif., USA). In another embodiment, the antibody is a CD36 Detection Antibody Clone 4H7 from Origene, such as TA700031, TA600031, and/or TA600044. Other suitable monoclonal antibodies from Origene include TA400016, CD36 mouse monoclonal antibody, clone 3F4, WB; TA400017, CD36 mouse monoclonal antibody, clone 3F4, WB; TA400020, CD36 mouse monoclonal antibody, clone 4H7, WB; TA400021, CD36 mouse monoclonal antibody, clone 4H7, WB; TA500921, CD36 mouse monoclonal antibody, Clone 3F4, WB, IHC, IF; TA500935, CD36 mouse monoclonal antibody, Clone 4H7, WB, IF, FC; TA500954, CD36 mouse monoclonal antibody, clone 6A5, WB, IHC; TA506366, CD36 mouse monoclonal antibody, Clone 1B3, WB, IHC, IF; TA600031, CD36 mouse monoclonal antibody, Clone 3F4, ELISA, AP; TA600044, CD36 mouse monoclonal antibody, Clone 6A5, ELISA, AP; and TA700031, CD36 mouse monoclonal antibody, Clone 4H7, ELISA, AP.

The antibodies used in the ELISA assay for detection of circulating CD36 in plasma are preferably labelled for ease of detection, the label preferably being in the form of a biotinylation; i.e. in a preferred embodiment, the antibody is biotinylated.

In particular the CD36-specific monoclonal antibody may be selected from the group consisting of:

### 185-1G2 Vilella

### 131.1 Tandon, Rockville

### 131.2 Tandon, Rockville

### 131.4 Tandon, Rockville

### 131.5 Tandon, Rockville

### 131.7 Tandon, Rockville

### NAM28-8C12 Blanchard, Nantes

### AmAK-5 Kehrel, Muenster

### CLB-IVC7 CLB, Amsterdam

### Lyp 10.5 McGregor, Lyon

### Lyp 13.10 McGregor, Lyon

### Standard Level Used in Diagnosis, Evaluation and Monitoring

In the provided methods for determining steatosis or steato-hepatitis or increased likelihood of having or developing steatosis or steato-hepatitis in an individual, for diagnosing steatosis or steato-hepatitis, monitoring the progression of steatosis or steato-hepatitis, evaluating the risk of acquiring steatosis or steato-hepatitis or developing more severe steatosis or steato-hepatitis and/or evaluating the effect of a treatment for steatosis or steato-hepatitis, an increased level of circulating CD36 polypeptide is typically associated with steatosis or steato-hepatitis, risk of acquiring steatosis or steato-hepatitis and/or more severe steatosis or steato-hepatitis. Thus, the concentration of circulating CD36 polypeptide or part thereof determined in a patient is correlated to a predetermined standard level, and any level of circulating CD36 polypeptide above such a predetermined standard level is indicative of steatosis or steato-hepatitis or increased likelihood of having or developing steatosis or steato-hepatitis.

In one embodiment, a level of soluble CD36 above the average level in a population of human beings is indicative of steatosis or steato-hepatitis, increased risk of having or acquiring steatosis or steato-hepatitis and/or more severe steatosis, such as steato-hepatitis.

Steatosis or steato-hepatitis may also be evaluated on the basis of the level of circulating CD36 with respect to the quartiles of a standard population. The quartiles of a set of values are the three points that divide the data set into four equal groups, each representing a fourth of the population being sampled. The first quartile (25^{th} percentile) cuts off the lowest 25% of the population; the second quartile (50^{th} percentile or median) cuts the population in half, and the third quartile (75^{th} percentile) cuts off highest 25% or lowest 75% of the population.

In one embodiment, a level of soluble/circulating CD36 above the sCD36 level of the first (25^{th} percentile) quartile of a population of human beings is indicative of steatosis or steato-hepatitis, increased risk of having or acquiring steatosis or steato-hepatitis and/or more severe steatosis or steato-hepatitis. In preferred embodiment, a level of soluble/circulating CD36 above the sCD36 level of the second quartile (50^{th} percentile) of a population of human beings is indicative of steatosis or steato-hepatitis, increased risk of having or acquiring steatosis or steato-hepatitis and/or more severe steatosis or steato-hepatitis. In a more preferred embodiment, a level of soluble/circulating CD36 above the sCD36 level of the third quartile (75^{th} percentile) of a population of human beings is indicative of steatosis or steato-hepatitis, increased risk of having or acquiring steatosis or steato-hepatitis and/or more severe steatosis or steato-hepatitis.

In another embodiment, an increase in the concentration of CD36 polypeptide of at least 1.15 times of the standard level is an indication of steatosis or steato-hepatitis, increased risk of having or acquiring steatosis or steato-hepatitis and/or more severe steatosis or steato-hepatitis. In particular an increase of at least 1.25 of the standard level, for example an increase of at least 1.30, such as increase of at least 1.35 of the standard level, for example 1.40 of the standard level, such as increase of at least 1.45 of the standard level, for example 1.50 of the standard level, such as an increase of at least 1.55 of the standard level, such as an increase of at least 1.60 of the standard level, such as an increase of at least 1.65 of the standard level, such as an increase of at least 1.70 of the standard level, such as increase of at least 1.75 of the standard level, such as an increase of at least 1.80 of the standard level, such as an increase of at least 1.85 of the standard level, such as an increase of at least 2.0 of the standard level, such as an increase of at least 2.25 of the standard level, such as an increase of at least 2.50 of the standard level, such as an increase of at least 3 of the standard level, such as an increase of at least 3.50 of the standard level, such as an increase of at least 4 of the standard level, such as an increase of at least 4.50 of the standard level, such as an increase of at least 5 of the standard level, such as an increase of at least 6, 7, 8, 9 or 10 of the standard level is indicative of steatosis or steato-hepatitis, increased risk of having or acquiring steatosis or steato-hepatitis and/or more severe steatosis or steato-hepatitis.

In an individual suffering from chronic hepatitis, such as chronic hepatitis C, such as genotype 1, an increase in the concentration of CD36 polypeptide of at least 1.15 times of the standard level is an indication of steatosis or steato-hepatitis, increased risk of having or acquiring steatosis or steato-hepatitis and/or more severe steatosis or steato-hepatitis. In particular an increase of at least 1.25 of the standard level, for example an increase of at least 1.30, such as increase of at least 1.35 of the standard level, for example 1.40 of the standard level, such as increase of at least 1.45 of the standard level, for example 1.50 of the standard level, such as an increase of at least 1.55 of the standard level, such as an increase of at least 1.60 of the standard level, such as an increase of at least 1.65 of the standard level, such as an increase of at least 1.70 of the standard level, such as increase of at least 1.75 of the standard level, such as an increase of at least 1.80 of the standard level, such as an increase of at least 1.85 of the standard level, such as an increase of at least 2.0 of the standard level, such as an increase of at least 2.25 of the standard level, such as an increase of at least 2.50 of the standard level, such as an increase of at least 3 of the standard level, such as an increase of at least 3.50 of the standard level, such as an increase of at least 4 of the standard level, such as an increase of at least 4.50 of the standard level, such as an increase of at least 5 of the standard level, such as an increase of at least 6, 7, 8, 9 or 10 of the standard level is indicative of steatosis or steato-hepatitis, increased risk of having or acquiring steatosis or steato-hepatitis and/or more severe steatosis or steato-hepatitis.

In an individual suffering from steatosis or steato-hepatitis, an increase in the concentration of CD36 polypeptide of at least 1.15 times of the standard level is an indication of increased risk of acquiring more severe steatosis or steato-hepatitis. In particular an increase of at least 1.25 of the standard level, for example an increase of at least 1.30, such as increase of at least 1.35 of the standard level, for example 1.40 of the standard level, such as increase of at least 1.45 of the standard level, for example 1.50 of the standard level, such as an increase of at least 1.55 of the standard level, such as an increase of at least 1.60 of the standard level, such as an increase of at least 1.65 of the standard level, such as an increase of at least 1.70 of the standard level, such as increase of at least 1.75 of the standard level, such as an increase of at least 1.80 of the standard level, such as an increase of at least 1.85 of the standard level, such as an increase of at least 2.0 of the standard level, such as an increase of at least 2.25 of the standard level, such as an increase of at least 2.50 of the standard level, such as an increase of at least 3 of the standard level, such as an increase of at least 3.50 of the standard level, such as an increase of at least 4 of the standard level, such as an increase of at least 4.50 of the standard level, such as an increase of at least 5 of the standard level, such as an increase of at least 6, 7, 8, 9 or 10 of the standard level is indicative of increased risk of acquiring more severe steatosis or steato-hepatitis.

By the term “standard level” is meant the concentration of CD36 polypeptide in a population, e.g. in a pool of samples from a random group of individuals. The population may be a random group of individuals, with or without any clinical symptoms with respect to steatosis or steato-hepatitis. However, in one embodiment, the population is a group of individuals, who have been diagnosed with hepatitis, such as chronic hepatitis C, in particular chronic hepatitis C genotype 1. In one embodiment the standard level is determined from a pool of samples from a random group of individuals. The pool comprises samples from at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or such as at least 100 individuals. Patients having CD36 levels in the area of a predetermined level determined for steatosis patients or steato-hepatitic patients can be inferred to have steatosis or steato-hepatitis, or be at risk of having or acquiring steatosis or steato-hepatitis.

In another embodiment the predetermined standard level is the concentration of CD36 polypeptide in a pool of samples from a group of individuals such as at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or such as at least 100 individuals, who has not been diagnosed with steatosis or steato-hepatitis, and in which no symptoms related to steatosis or steato-hepatitis have been observed more than 2 months ago, more than 3 months ago, more than 4 months ago, more than 5 months ago, or more than 6 months ago at the time for supplying a sample. Such a predetermined standard level can be used to identify patients having elevated levels of CD36 polypeptide or part thereof, because these patients have levels of CD36 above a predetermined level determined for healthy/non steato-hepatitic patients.

### EXAMPLES

### Example 1

### Example of ELISA Analysis Kit for Detecting CD36 in Serum

ELISA analysis, reagents used and assay conditions
Phosphate buffer 0.1 mol/l, pH 8.0
Na₂HPO4, 2H₂O, 0.1 mol/l, pH 8.0, stored at 4° C.

### POD Buffer

NaH₂PO4, H₂O, 1.5 mmol/l
Na₂HPO4, 2 H₂O, 8.5 mmol/l
NaCl 400 mmol/l
pH 7.4

### Stored at 4° C. 20

### Preparation of Lysozyme Solution:

Lysozyme, Sigma L-6876, 20 mg/ml. Stored at −20° C. in 1 ml portions, storage life 1 year. Storage life at 4° C. is 4 days.

### POD-Avidin Dako P364: Colour Reagent

TMB Microwell Peroxidase Substrate (1 component)
Cat. No. 50-76-06, Kirkegård and Perry Laboratories. Storage life 1 year at 2-25° C. 25
Phosphoric Acid, 1 mol/l

### POD-Avidin Solution:

POD buffer 12 ml
Lysozyme solution 120 μl 30

### POD-avidin 6 μl

To be prepared immediately before use.

### Antibody

Anti-CD36: biotinylated sc-9154 (rabbit polyclonal antibody from Santa Cruz)

### Apparatus

Automatic microtiter plate-rinser Elx50 (Biotek Instruments).

ELISA plates were coated overnight at 4° C. with catcher antibody goat (sc5522)—CD36 (0.1 μg/ml), followed by blocking with phosphate-buffered saline (PBS) and 0.1% Tween for 2 days at 4° C. The plates were stored at −20° C. until use. A microtiter plate coated with antibody against CD36 is rinsed 3 times with 340 μl rinsing buffer pr. well using program 12. The last rinse is terminated without aspiration of rinsing buffer, which is first decanted immediately before application of standards (EDTA-plasma pool) and samples (with possible controls), and immediately before addition of reagent. 5

### Standards

EDTA blood from 100 subjects from the routine blood sampling are centrifuged at 1890 G for 10 min, the upper part of the plasma is pipetted off and pooled. Pool-plasma is frozen in aliquots of 350 μl and dilutions are used as standard curves. Other EDTA plasma pools are used as high and low internal assay controls.

The intra assay coefficient of variation, which was estimated from a run of 76 single determinations of the same sample, was 10%, and estimated from double determinations of the high control on 15 different days was 6%. The inter assay coefficient of variation, estimated from the controls in each run performed, was 16.4%. Runs were only accepted when controls were within the range of +/−1.96×SD (inter assay).

### Method for ELISA Assay

A microtiter plate is rinsed on the Elx50. Standards, controls, samples and dilution buffer are applied in double rows, 100 μl/well. The positions of the applications are noted. The micro-titer plate is covered with plastic film and incubated for 60 min. on a shaking table. The plate is rinsed on the Elx50. 100 μl biotinylated sc-9154 is added per well, covered with plastic film and incubate 60 min on a shaking table. The plate is rinsed on the Elx50. 100 μl POD-avidin solution is added per well. Covered with plastic film and incubate for about 30 min. on a shaking table. The plate is rinsed on the Elx50. In a fume hood 100 μl TMB is added per well. Cover with plastic film, incubate for about 10 min. on a shaking table. The reaction is terminated with 100 μl phosphoric acid per well (in a fume hood). Cover with plastic film until reading.

### Measurement

Read the extinctions at 450 nm and 620 nm on a Multiscan apparatus before 60 min following the termination of the reaction.

### Calculation

Cubic spline with linear scale on both axes. Dilutions of EDTA pool are used as standard curve, and results are expressed relative to the EDTA plasma pool.

Elisa dilution buffer: phosphate buffer 10 mmol/l with 0.145 mol/l NaCl, pH 7.4

### Example 2

Plasma sCD36 is associated with markers of atherosclerosis, insulin resistance and fatty liver in a non-diabetic healthy population

This example demonstrates the relationships between plasma sCD36 and measures of insulin resistance, fatty liver and carotid atherosclerosis in a large population of healthy subjects.

Abbreviations Used in this Example:
sCD36: soluble CD36; MetSy: Metabolic syndrome; T2D: Type 2 diabetes; CVD: Cardiovascular disease; oxLDL: oxidized low density lipoprotein; PCOS: Polycystic ovary syndrome; BP: Blood pressure; FPG: Fasting plasma glucose; FFM: Fat-free mass; LDL: LDL-cholesterol; HDL: HDL-cholesterol; FFA: Free fatty acids; GGT: Gamma-glutamyltransferase; ALT: Alanine aminotransferase; AST: Aspartate aminotransferase; M/I: insulin sensitivity; FLI: Fatty liver index; MRS: magnetic resonance spectroscopy; BMI: Body mass index; LF %: Liver fat percentage; IMT: intima-media thickness; OGTT: Oral glucose tolerance test; NGT: Normal glucose tolerance; IFG: Impaired fasting glucose; IGT: Impaired glucose tolerance; +MetSy: presence of the metabolic syndrome; −MetSy: Absence of metabolic syndrome; Log-normal population: subjects with sCD36 within the log-normal distribution;

### Experimental Methods

### Study Subjects

The RISC study is a prospective, observational, cohort study. In brief, clinically healthy Caucasian men and women, aged between 30-60 years, were recruited from 19 centres in 14 European countries. Initial exclusion criteria were: treatment for obesity, hypertension, lipid disorders or diabetes, pregnancy, cardiovascular or chronic lung disease, weight change of ≧5 kg in last 6 months, cancer (in last 5 years) and renal failure. Exclusion criteria after screening were: systolic and diastolic blood pressure (BP)≧140/90 mmHg; fasting plasma glucose (FPG)≧27.0 mmol/1; 2-hour plasma glucose ≧11.0 mmol/l; total serum cholesterol ≧7.8 mmol/l; serum triglycerides ≧4.6 mmol/l; ECG abnormalities and carotid artery plaques. The present analysis is based on the 1296 subjects (712 women and 584 men), who satisfied all criteria, had plasma sCD36 measured, and whose clamp study (see below) passed the quality control check. Local Ethics Committee approval was obtained by each recruiting centre.

### Physical Examinations and Lifestyle Factors

Height, body weight, percent body fat, fat-free mass (FFM), waist circumference, sitting BP and heart rate were measured as previously described. A lifestyle questionnaire was used to collect information on smoking and alcohol habits.

### Analytical Determinations

Blood collected during the studies was separated into plasma and serum, aliquoted, and stored at −20° C. for glucose and −80° C. for lipids, insulin and sCD36. Plasma glucose, serum insulin, plasma adiponectin, LDL-cholesterol (LDL), HDL-cholesterol (HDL), triglycerides, free fatty acids (FFA), gamma-glutamyltransferase (GOT), aspartate aminotransferase (AST), and alanine aminotransferase (ALT) were all measured in a central laboratory, as described.

Plasma Soluble CD36 (sCD36)

sCD36 was measured using an in-house ELISA-assay on Li-Heparin plasma [Handberg A, Levin K, Hojlund K, Beck-Nielsen H. Identification of the oxidized low-density lipoprotein scavenger receptor CD36 in plasma: a novel marker of insulin resistance. Circulation 2006; 114(11): 1169-76]. A pool of EDTA plasma was applied in 7 dilutions and used as a standard concentration curve. Internal controls were run in quadruplicates on each plate. Runs were accepted if the controls were within ±2 SD from mean, and most were within 1 SD. Intra-assay CV % was 6%, and total day-to-day-assay CV % was 16.4%. Log-transformed standard curves were linear. Few measurements outside standard curve range were calculated by extrapolation. sCD36 was measured on fasting samples. Detection limit established as zero calibrator plus 2 SD of the lowest calibrator (N=48) was 0.027 arbitrary units in this study.

### Insulin Clamp.

An euglycaemic hyperinsulinaemic clamp was performed in all subjects. Insulin sensitivity was expressed as the ratio of the M value—averaged over the final 40 min of the 2-hour clamp and normalized by FFM—to mean plasma insulin concentration during the same interval (M/I, in units of μmol·min⁻¹·kg FFM⁻¹·nmol/l⁻¹).

### Fatty Liver Index

The fatty liver index (FLI) uses an algorithm based on body mass index (BMI), waist circumference, triglycerides and GGT with an accuracy of 0.84 (95% confidence interval 0.81-0.87) in detecting fatty liver. In the present example, we have used restrictive cut points (FLI<20 and FLI>60), for which we have previously estimated the likelihood to have or not have fatty liver by the Bayes theorem on the RISC study cohort; when the index value (FLI) is greater than 60 the likelihood to have fatty liver is greater than 78%, if FLI is less than 20 the likelihood not to have fatty liver is greater than 91%. Subjects were then categorized in three groups: FLI<20, FLI: 20-60, and FLI>60. To validate the use of FLI in the RISC cohort, we have evaluated the hepatic fat content in a separate group of subjects (n=37) by magnetic resonance spectroscopy (MRS) and compared with our estimated cut off points for the likelihood to have fatty liver on the basis on FLI values. Subjects with FLI>20 had no hepatic fat content (range 0.4-4.2%, n=6), while those with FLI>60 had steatosis with hepatic fat content >5% (range 8.6%-24%, n=10). Moreover, FLI correlated significantly with hepatic fat content determined by magnetic resonance spectroscopy (MRS) (r=0.61, p=0.0001).

### Liver Fat Percentage (LF %)

Liver fat percentage (LF %) uses an algorithm based on presence of MetSy and T2DM as well as fasting insulin, aspartate- and alanine-aminotransferases to predict the degree of liver fat accumulation. The algorithm was developed and validated by H-MRS and moreover, the algorithm was validated in a separate study group (Kotronen A, Peltonen M, Hakkarainen A, Sevastianova K, Bergholm R, Johansson L M et al. Prediction of non-alcoholic fatty liver disease and liver fat using metabolic and genetic factors. Gastroenterology 2009; 137(3): 865-72). The R² of the LF % algorithm against ¹H-MRS was 0.49.

### Carotid Intima-Media Thickness (IMT) Analysis

We followed a validated scanning and reading protocol for ultrasound measurement of carotid artery IMT. The IMT value was computed as an average of all carotid walls available (up to 4 walls). Infra-observer variability of IMT measures was tested in 100 randomly chosen scans calculating the average percent difference between the first and second reading relative to the first reading. The mean difference was 4.6±3.0%.

### Glucose Tolerance and MetSy.

Participants underwent a 2-hour standard 75 g oral glucose tolerance test (OGTT) and classified into normal (NGT), impaired fasting glycaemia (IFG) and impaired glucose tolerance (IGT) according to American Diabetes Association criteria from 2000. Subjects with IFG and/or IGT were pooled as impaired glucose regulation (n=182 or 14% of the total cohort). The presence (+MetSy) or absence (−MetSy) of the metabolic syndrome was defined according to the criteria from the International Diabetes Federation.

### Statistical Analysis

All data analyses were performed with the SPSS 11.0 for Windows program (SPSS, Inc., Chicago, Ill.). The results for continuous variables are reported as the mean±SD unless otherwise is stated and for classes as %. Variables with skewed distribution (sCD36, insulin, triglycerides. FFA, adiponectin, ALT, GGT, FLI, LDH, HDL, M/I, and IMT) were logarithmically transformed for statistical analyses. One-way ANOVA and Tukey's post hoc analysis was used to analyze the markers of insulin resistance, carotid atherosclerosis and fatty liver by quartiles (Q1 to Q4) of sCD36. Chi square tests were used for binominal variables (MetSy and IGT/IFG). Partial correlation analyses were used to test the relationship between sCD36 and the same markers when adjusted for centre, gender, glucose tolerance status, smoking habits and alcohol consumption. P≦0.05 was considered significant.

### Results

Effect of Gender, Glucose Tolerance, ±MetSy and FLI on sCD36

Plasma sCD36 was significantly lower in women than in men, like other indices of insulin resistance, atherosclerosis and fatty liver, such as fasting glucose, insulin, LDL-cholesterol, triglycerides, ALT, GGT, FLI, LF %, BMI, waist, clamp FFA, IMT and diastolic and systolic BP, while insulin sensitivity (M/I), plasma adiponectin and HDL were significantly higher (Table 1). Accordingly, the presence of the MetSy, but not IGT/IFG, was lower in women. In contrast (with respect to direction of parameters, which have previously been associated with insulin sensitivity), fasting FFA, age, and percent body fat were significantly higher in women than in men.

TABLE 1
Clinical and laboratory characteristics of the RISC Cohort

| | All | Male | Female | P value |
|---|---|---|---|---|
| N (1276-1296) | 1296 | 584 | 712 | |
| Age (yr) | 43.9 ± 8.3 | 43.2 ± 8.5 | 44.4 ± 8.1 | 0.010 |
| Waist (cm) | 87 ± 13 | 93 ± 10 | 81 ± 12 | <0.001 |
| Percent body fat (%) | 20.9 ± 8.8 | 19.0 ± 7.9 | 22.6 ± 9.1 | <0.001 |
| BMI (kg/m²) | 25.5 ± 4.0 | 26.4 ± 3.5 | 24.8 ± 4.3 | <0.001 |
| Diastolic BP (mmHg) | 74 ± 8 | 76 ± 7 | 73 ± 8 | <0.001 |
| Systolic BP (mmHg) | 117 ± 12 | 122 ± 11 | 114 ± 13 | <0.001 |
| Heart rate | 68 ± 10 | 66 ± 10 | 70 ± 10 | <0.001 |
| LDL (mmol/l)* | 2.9 ± 0.8 | 3.1 ± 0.8 | 2.8 ± 0.8 | <0.001 |
| HDL (mmol/l)* | 1.4 ± 0.4 | 1.2 ± 0.3 | 1.6 ± 0.4 | <0.001 |
| Triglyceride (mmol/l)* | 1.1 ± 0.7 | 1.3 ± 0.9 | 0.9 ± 0.5 | <0.001 |
| Fasting FFA (mmol/l)* | 0.538 ± 0.228 | 0.474 ± 0.222 | 0.590 ± 0.219 | <0.001 |
| Clamp FFA (mmol/l)* | 0.054 ± 0.110 | 0.063 ± 0.099 | 0.048 ± 0.118 | <0.001 |
| Fasting glucose (mmol/l) | 5.1 ± 0.5 | 5.2 ± 0.5 | 4.9 ± 0.5 | <0.001 |
| Fasting insulin (pmol/l)* | 32 ± 21 | 35 ± 25 | 30 ± 18 | <0.001 |
| GGT (U/l)* | 25.9 ± 19.0 | 32.9 ± 24.2 | 21.7 ± 20.3 | <0.001 |
| ALT (U/l)* | 20.1 ± 11.6 | 24.8 ± 13.4 | 16.4 ± 8.1 | <0.001 |
| FLI* | 27.8 ± 25.0 | 38.5 ± 25.0 | 18.9 ± 21.2 | <0.001 |
| LF %* | 2.04 ± 1.44 | 2.47 ± 1.73 | 1.69 ± 1.04 | <0.001 |
| Plasma adiponectin (mg/l)* | 8.4 ± 3.8 | 6.5 ± 2.6 | 9.9 ± 3.8 | <0.001 |
| M/I | 141 ± 72 | 124 ± 64 | 155 ± 75 | <0.001 |
| (μmol · min⁻¹ · kg_{FFM}⁻¹ · nmol/l⁻¹)* | | | | |
| Carotid IMT (mm)* | 0.60 ± 0.09 | 0.62 ± 0.09 | 0.59 ± 0.08 | <0.001 |
| +MetSy* | 13.70 ± 34.40 | 18.58 ± 38.93 | 9.70 ± 29.62 | <0.001 |
| IGT/IGF | 14.06 ± 34.78 | 15.44 ± 36.16 | 12.94 ± 33.59 | 0.20 |
| Plasma CD36* | 3.1 ± 3.3 | 3.3 ± 3.5 | 2.9 ± 3.2 | 0.045 |

Mean ± SD,
*P-values based on statistical analyses using log-normalized values.

Quartiles of Plasma sCD36

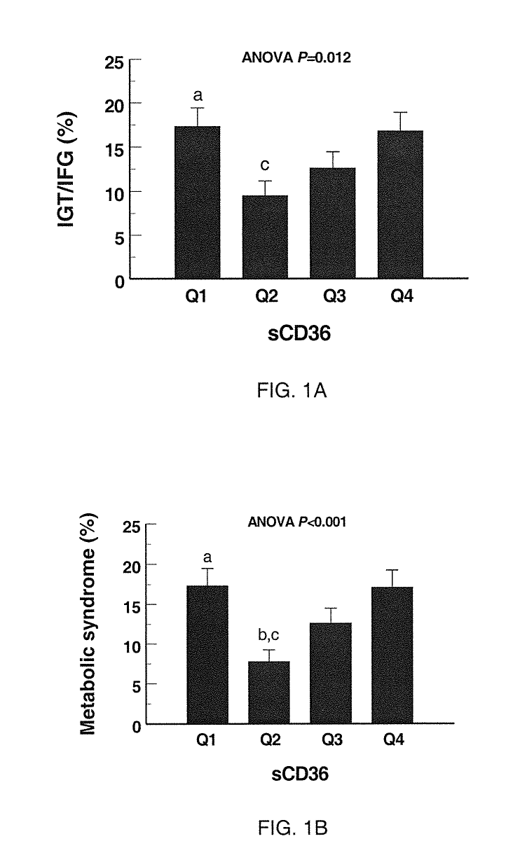

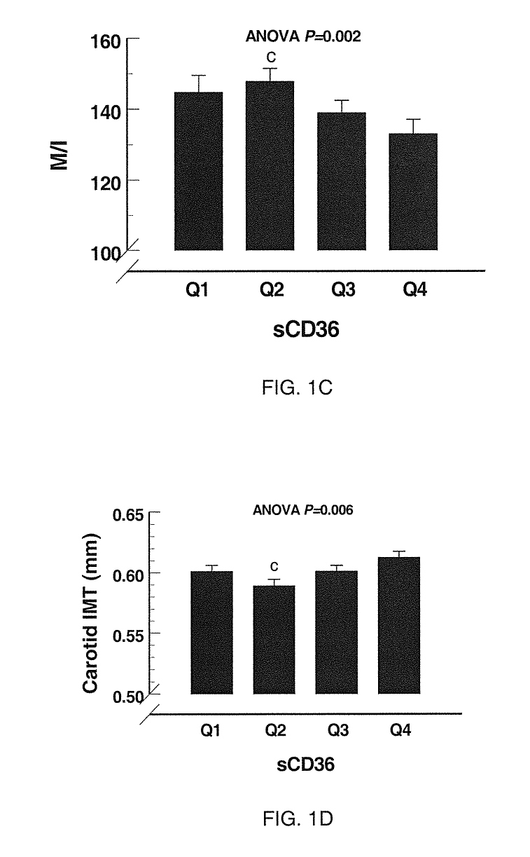

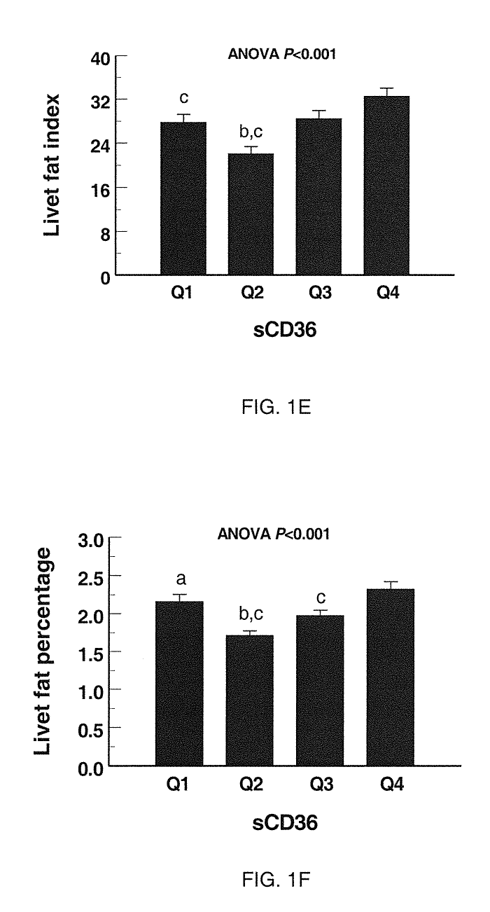

HDL and M/I decreased across quartiles (Q2, Q3 and Q4) of plasma log sCD36, whereas FPG, insulin, triglycerides, LDL, clamp FFA, age, waist, percent body fat, ALT, GGT, FLI, LF %, BMI, systolic BP, IMT, IGT/IFG and +MetSy increased across the same quartiles (ANOVA; p<0.01 for all) (Table 2, FIG. 1). Plasma adiponectin decreased across all four quartiles. For all these parameters, the largest difference was observed between Q2 and Q4 values of plasma sCD36, and for all variables, except ALT, this difference was significant (Table 2). Most variables exhibited a J-shaped (or inverse J-shaped) relationship with sCD36, with Q1 values showing the opposite trend as observed through Q2->Q3->Q4. Waist, percent body fat, BMI, ALT, GGT, LF %, +MetSy and IGT/IFG showed significant difference between Q1 and Q2 (Table 2, FIG. 1) whereas LFI, M/I, IMT and the remainder in Table 2 did not reach statistical significance. In both men and women fasting insulin, waist, FLI, LF %, +MetSy and percent body fat increased across sex-specific quartiles (Q2, Q3 and Q4) of sCD36, whereas adiponectin decreased across all four quartiles (ANOVA; p<0.05 for all (data not shown). In women, significant differences across quartiles of plasma sCD36 were seen for age, BMI, fasting glucose, triglycerides, HDL, systolic BP and IGT/IFG (p<0.02 for all), whereas in men changes across quartiles of sCD36 were seen for GGT (p=0.021) and LDL (p=0.005).

TABLE 2
Relationship between sCD36 and anthropometric measures and biochemical markers
of insulin resistance and fatty liver in the RISC cohort.

| | Plasma sCD36 | | | | P |
|---|---|---|---|---|---|
| | Q1 | Q2 | Q3 | Q4 | all |
| | <0.8 | 0.8-2.3 | 2.3-4.3 | >4.3 | |
| | 323 | 325 | 325 | 323 | |
| Age (yr) | 43.3 ± 8.2^{c} | 43.4 ± 8.3^{c} | 43.2 ± 8.1^{c} | 45.4 ± 8.4 | 0.002 |
| Waist (cm) | 86 ± 13^{a} | 83 ± 12^{b,c} | 88 ± 12 | 89 ± 12 | <0.001 |
| Percent body fat | 21.7 ± 9.5^{a} | 19.4 ± 8.0^{c} | 20.6 ± 8.9 | 22.1 ± 8.5 | <0.001 |
| (%) | | | | | |
| BMI (kg/m²) | 25.8 ± 4.4^{a} | 24.6 ± 3.7^{b,c} | 25.5 ± 3.9 | 26.2 ± 4.0 | <0.001 |
| Diastolic BP (mmHg) | 74 ± 8 | 74 ± 8 | 75 ± 8 | 75 ± 8 | 0.143 |
| Systolic BP (mmHg) | 117 ± 13 | 116 ± 12^{c} | 117 ± 13 | 119 ± 12 | 0.034 |
| Heart rate | 67 ± 10 | 68 ± 11 | 69 ± 10 | 69 ± 10 | 0.085 |
| LDL (mmol/l)* | 2.9 ± 0.8^{c} | 2.8 ± 0.8^{c} | 2.9 ± 0.8^{c} | 3.1 ± 0.8 | <0.001 |
| HDL (mmol/l)* | 1.4 ± 0.4 | 1.5 ± 0.4^{b,c} | 1.4 ± 0.4 | 1.4 ± 0.4 | <0.001 |
| Triglyceride | 1.1 ± 0.9^{c} | 1.0 ± 0.6^{c} | 1.1 ± 0.6 | 1.2 ± 0.7 | <0.001 |
| (mmol/l)* | | | | | |
| Fasting FFA | 0.53 ± 0.21 | 0.55 ± 0.22 | 0.54 ± 0.27 | 0.53 ± 0.22 | 0.528 |
| (mmol/l)* | | | | | |
| Clamp FFA | 60 ± 164^{c} | 48 ± 70 | 52 ± 81 | 58 ± 99 | 0.022 |
| (nmol/l)* | | | | | |
| Fasting glucose | 5.1 ± 0.5 | 5.0 ± 0.5^{c} | 5.1 ± 0.6 | 5.1 ± 0.6 | 0.008 |
| (mmol/l) | | | | | |
| Fasting insulin | 32 ± 25^{c} | 28 ± 17^{c} | 31 ± 19^{c} | 38 ± 22 | <0.001 |
| (pmol/l)* | | | | | |
| ALT (U/l)* | 21.4 ± 12.8^{a} | 18.8 ± 12.4 | 20.2 ± 10.2 | 20.5 ± 10.6 | 0.016 |
| GGT (U/l)* | 27.4 ± 23.0^{a} | 24.5 ± 24.7^{c} | 25.2 ± 16.4^{c} | 30.0 ± 25.4 | <0.001 |
| Plasma adiponectin | 9.2 ± 3.9^{b,c} | 8.7 ± 3.9^{b,c} | 7.9 ± 3.5 | 7.8 ± 3.5 | <0.001 |
| (mg/l)* | | | | | |

Mean ± SD,
*P-values based on statistical analyses of log-normalized values.
^{a}P < 0.05 vs. Q2,
^{b}P < 0.05 vs. Q3, and
^{c}P < 0.05 vs. Q4 in post-hoc analysis.

Bi-Modal Distribution of sCD36—Two Populations

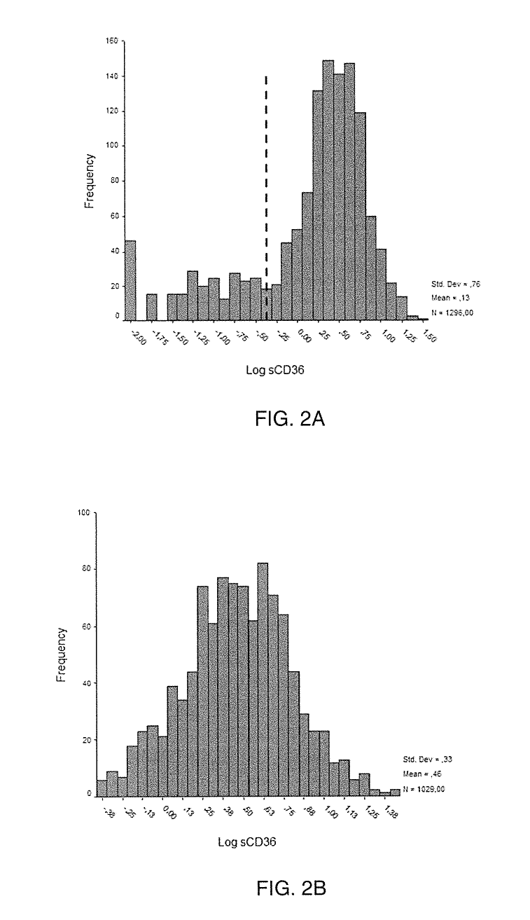

The J-shaped relationship between most variables and quartiles of sCD36 suggested the possible existence of two populations of sCD36 values. A closer examination at the log-transformed distribution of sCD36 revealed that a part of the total population had extremely low values of sCD36 (FIG. 2). It was evident that log values of sCD36 less than −0.38 do not belong to the log normal distribution of sCD36. Excluding those with log sCD36<-0.38, we observed in the resulting Log-normal population significantly higher plasma sCD36 levels in men vs women (4.1±3.5 vs. 3.7±3.1; p=0.001), IGT/IFG vs. NGT subjects (4.5±4.0 vs. 3.8±3.2; p=0.045), and in +MetSy vs. −MetSy subjects (4.6±3.9 vs. 3.8±3.2; p=0.006). The differences in glucose tolerance status and presence of MetSy were also significant in women (p<0.05), but not in men. Moreover, plasma sCD36 levels increased significantly with increasing likelihood of fatty liver (FLI<20->FLI 20-60->FLI>60) (3.5±3.1 vs. 4.2±3.5 vs 4.6±3.4; p<0.001). After post-hoc analysis, plasma sCD36 was significantly lower in subjects with FLI<20 than in subjects with FLI 20-60 (p=0.009) and FLI>60 (p=0.009).

It was also tested whether the characteristics of the two populations of sCD36 distribution differed from each other, but no differences were observed in the parameters shown in Table 1 except for significantly higher levels of plasma adiponectin (9.00±3.89 vs. 8.22±3.70 mg/l; p<0.001), FFA clamp (0.062±0.179 vs 0.052±0.084 mmol/l; p<0.028) and increased presence of MetSy (18.3±38.8 vs. 12.5±33.1; p<0.027) in the population with log sCD36<-0.38 (n=267). Of note, the higher plasma adiponectin levels were not caused by a higher ratio of women to men (56% vs 55%; p=0.75).

### Correlation Analyses

In the total population, univariate analysis revealed significant correlations between plasma sCD36 and fasting insulin, triglycerides, adiponectin, clamp FFA and FLI (Table 3). After adjustment for centre, gender, age, glucose tolerance status, smoking habits and alcohol consumption using multivariate analysis, these associations remained significant. In addition, it introduced an inverse relationship between WI and sCD36. The sCD36 log-normal population (log sCD36≧−0.38) revealed stronger or similar associations between sCD36 and fasting insulin, adiponectin, triglycerides, clamp FFA, GGT and FLI, and introduced significant associations of sCD36 with fasting glucose, LDL, HDL, +MetSy, LF % and carotid IMT (Table 3). Except for fasting glucose, adiponectin and +MetSy, these associations persisted after adjusting for centre, gender, age, glucose tolerance status, smoking habits and alcohol consumption. Univariate or multivariate analysis of sCD36 and the variables shown in Table 3 within the population with log sCD36<-0.38 showed no significant associations.

A strong positive correlation between the two estimates of fatty liver, FLI and LF %, was observed both in the total population (r=0.66; p<0.001) and in the sCD36 log-normal population (r=0.64; p<0.001).

TABLE 3
Univariate and multivariate analyses of the relationship between sCD36
and markers of insulin resistance, carotid atherosclerosis and fatty liver
in the RISC Cohort

| | | | Population with log | |
|---|---|---|---|---|
| | Total population | | sCD36 ≧ −0.38 | |
| | (n = 1296) | | (n = 1029) | |
| | Univariate | Multivariate | Univariate | Multivariate |
| | analysis | analysis | analysis | analysis |
| LDL (mmol/l)^{a} | 0.049 | 0.040 | 0.131** | 0.087** |
| HDL (mmol/l)^{a} | −0.013 | −0.019 | −0.103** | −0.102** |
| Triglyceride | 0.066* | 0.067* | 0.149** | 0.112** |
| (mmol/l)^{a} | | | | |
| clamp FFA | 0.086** | 0.075** | 0.066* | 0.094** |
| (mmol/l)^{a} | | | | |
| Fasting glucose | −0.007 | −0.016 | 0.113** | 0.015 |
| (mmol/l) | | | | |
| Fasting insulin | 0.091** | 0.086** | 0.180** | 0.126** |
| (pmol/l)^{a} | | | | |
| ALT (U/l)^{a} | −0.020 | 0.019 | 0.049 | 0.023 |
| GGT (U/l)^{a} | 0.025 | 0.027 | 0.152** | 0.101** |
| FLI^{a} | 0.095** | 0.094** | 0.184** | 0.114** |
| LF %^{a} | 0.031 | 0.041 | 0.151** | 0.102** |
| Plasma adiponectin | −0.118** | −0.067* | −0.115** | −0.048 |
| (mg/l)^{a} | | | | |
| M/I | −0.041 | −0.069* | −0.051 | −0.072* |
| (μmol · min⁻¹ · | | | | |
| kg_{FFM}⁻¹ · nmol/l⁻¹)^{a} | | | | |
| No of factors in | 0.003 | 0.023 | 0.104** | 0.059 |
| the metabolic | | | | |
| syndrome | | | | |
| Carotid IMT | 0.022 | 0.047 | 0.104** | 0.085* |
| (mm)^{a} | | | | |

In multivariate analysis, partial correlations coefficients are given after adjustment for centre, age, gender, glucose tolerance status, smoking habits and alcohol consumption.
^{a}P-values based on statistical analyses of log-normalized values
*p < 0.05,
**p < 0.01

### Discussion

In this cross-sectional study of non-diabetic generally healthy subjects, plasma sCD36 showed significant associations with insulin resistance, carotid atherosclerosis and fatty liver. The association of sCD36 with a large range of risk factors of insulin resistance and CVD was consistently significant. At closer inspection, these associations tend to be J-shaped (or inverse J-shaped) for the majority of risk factors. Thus, both high and very low sCD36 in a general healthy population seem to carry an increased risk of disease. Several studies have reported an association between high CD36 expression and increased foam cell formation and unstable atherosclerosis. On the other hand, a study of the general phenotypic appearance of CD36 deficient patients reported that CD36 deficiency was associated with hyperlipidemia and an atherogenic lipid profile, insulin resistance, and mild hypertension. Thus, it is possible that both too much and too little sCD36 may be associated with an increased disease risk, at least in clinically healthy people. Low CD36 expression was associated with increased fatty acid flux to the liver, which may result in dyslipidemia and insulin resistance. High sCD36 was associated with insulin resistance, and in diabetic mouse and human monocytes, CD36 gene expression was upregulated as a consequence of impaired insulin signaling. In addition, impaired insulin signaling in an atherosclerosis prone mouse model was associated with elevated CD36 expression in circulating leukocytes. Furthermore, high oxLDL, as a consequence of increased oxidative stress and dyslipidemia, upregulates monocyte and macrophage CD36 expression, and may lead to accelerated atherosclerosis. Therefore our findings, that both high and low sCD36 may be associated with risk factors of insulin resistance and CVD, are not in conflict with the existing literature. Seeking other possible explanations, a mutation in the coding region of the CD36 gene may result in altered immunoreactivity with the antibodies used in the sCD36 ELISA assay, and this may lead to false low sCD36 levels. A CD36 promotor mutation in Caucasians linked to diabetes has been reported, and mutations in the coding area of the CD36 gene leading to CD36 deficiency in platelets and monocytes are rare in Caucasians. Another methodological explanation could be a matrix effect since low concentrations were mostly run undiluted in contrast to the remaining samples. Whatever cause for the distribution of sCD36, the log normal sCD36 population exhibited higher sCD36 in participants with disturbed glucose tolerance, with the MetSy, and with an increased likelihood of fatty liver disease in accordance with previous findings.

Targeted over-expression of CD36 in the liver causes liver fat accumulation in mice. In addition, diet induced obesity is associated with higher liver CD36 expression, liver fat accumulation and dyslipidemia (Koonen D P, Jacobs R L, Febbraio M, Young M E, Soltys C L, Ong H et al. Increased hepatic CD36 expression contributes to dyslipidemia associated with diet-induced obesity. Diabetes 2007; 56(12): 2863-71). So far, there are only a few studies on human liver CD36. In liver biopsies from NAFLD patients, fatty acid transporting protein expression, and among those CD36 expression, correlated with liver fat content, and it was concluded that in liver steatosis multiple genes related to lipid metabolism were involved. In a very recent study by Miquilena-Colina and coworkers subjects with fatty liver and chronic hepatitis C virus infection have increased CD36 expression in liver and significant associations between hepatic CD36 expression and insulin resistance as well as with degree of liver steatosis Circulating sCD36 is associated with indicators of liver function, such as ALAT, in insulin resistant subjects with IGT or T2D but not in subjects with normal glucose homeostasis, and it was hypothesized that sCD36 may be a marker of NAFLD. Here we find that soluble CD36 (sCD36) increases with increased likelihood of fatty liver, and furthermore, that this association between sCD36 and fatty liver was significant even after corrections for several confounders. The fact that sCD36 was also found to be associated with LF % in the sCD36 log-normal population, and that the variables used for estimation of the LF % are different from those in FLI support this hypothesis. One major limitation is that the derived measure, FLI, was developed in a general population using ultrasonography rather than magnetic resonance spectroscopy, which is the gold-standard technique for the diagnosis of non-alcoholic fatty liver disease. However, LF % is validated by magnetic resonance spectroscopy. Since there is a strong correlation between the two algorithms of fatty liver despite that they are derived from different variables, and since sCD36, being a fatty acid transporter of the liver, is correlated to both, it is reasonable to consider sCD36 to be a new liver fat marker.

In summary, this study shows that in the context of a cross-sectional study of generally healthy non-diabetic subjects sCD36 represents a new biomarker of a phenotype carrying insulin resistance, carotid atherosclerosis and fatty liver.

### Example 3

High sCD36 plasma level is associated with steatosis and its severity in patients with chronic genotype 1 hepatitis C

### Summary

In this example, soluble CD36 (sCD36) plasma levels were associated with surrogate marker of steatosis. In a cohort of patients with genotype 1 chronic hepatitis C (G1CHC), we tested the association of sCD36 plasma levels with host and viral factors and sustained virological response (SVR).

One hundred seventy-five consecutive biopsy-proven G1CHC patients were studied. sCD36 plasma levels were assessed by ELISA. All biopsies were scored by one pathologist for staging and grading, and graded for steatosis, which was considered moderate-severe if ≧20%. Patients underwent standard of care therapy with pegylated-interferon and ribavirin.

Results: A progressive increase in the severity of steatosis was found according to sCD36 quartiles (p=0.02); total and LDL cholesterol levels were significantly higher in patients in the lower quartile compared to all the others. Gamma-glutamyl transferase (p=0.02), HOMA score (p=0.002), and sCD36 (p=0.04) were independently associated with the severity of steatosis as continuous variable. Multivariate logistic regression analysis showed that HOMA (OR 1.243, 95% Cl 1.04-1.484, p=0.01) and sCD36 (OR 1.445, 95% Cl 1.135-1.839, p=0.003), were independently linked to steatosis ≧20%. No association was found between sCD36 and SVR.

These results demonstrate that CD36 have a role in the pathogenesis of steatosis and insulin resistance in patients with G1CHC, and is a suitable candidate marker for steatosis. These results are described in more detail herein below.

### Experimental Methods

### Patients

One-hundred and seventy-five consecutive patients with G1 CHC, recruited at the Gastrointestinal & Liver Unit at the University Hospital in Palermo and fulfilling all inclusion and exclusion criteria detailed below, were assessed. Patients were included if they had a histological diagnosis of CHC (any degree of fibrosis, including cirrhosis) on a liver biopsy performed within 6 months prior to enrolment. G1 CHC patients were characterized by the presence of anti-HCV and HCV RNA, with persistently abnormal alanine aminotransferase (ALT), and by alcohol consumption of <20 g/day in the last year or more, evaluated by a specific questionnaire. Exclusion criteria were: 1) advanced cirrhosis (Child-Pugh B and C); 2) hepatocellular carcinoma; 3) other causes of liver disease or mixed etiologies (excessive alcohol consumption, hepatitis B, autoimmune liver disease, Wilson's disease, hemochromatosis, α1-antitrypsin deficiency); 4) HIV infection; 5) previous treatment with antiviral therapy, immuno-suppressive drug and/or regular use of steatosis-inducing drugs (corticosteroids, valproic acid, tamoxifen, amiodarone); and 6) active IV drug addiction.

The study was performed in accordance with the principles of the Helsinki Declaration and its appendices, and with local and national laws. Approval was obtained from the hospital's Institutional Review Board and Ethics Committee, and written informed consent was obtained from all patients.

### Clinical and Laboratory Assessment

Clinical and anthropometric data were collected at the time of liver biopsy. Body mass index (BMI) was calculated on the basis of weight in kilograms and height (in meters), and patients were classified as normal weight (BMI, 18.5-24.9 kg/m²), overweight (BMI, 25-29.9), or obese (BMI≧30). Waist circumference (WC) was measured at the midpoint between the lower border of the rib cage and the iliac crest. The diagnosis of arterial hypertension was based on the following criteria: systolic blood pressure ≧130 mm Hg and/or diastolic blood pressure ≧85 mm Hg (measured three times within 30 minutes, in the sitting position and using a brachial sphygmomanometer), or use of blood-pressure-lowering agents. The diagnosis of type 2 diabetes was based on the revised criteria of the American Diabetes Association, using a value of fasting blood glucose ≧126 mg/dL on at least two occasions. In patients with a previous diagnosis of type 2 diabetes, current therapy with insulin or oral hypoglycemic agents was documented. The metabolic syndrome was diagnosed according to Adult Treatment Panel III criteria.

A 12-hour overnight fasting blood sample was drawn at the time of liver biopsy to determine serum levels of ALT, γ-glutamyl transferase, total cholesterol, HDL and LDL-cholesterol, triglycerides, plasma glucose concentration, insulin, and platelet count. Insulin resistance (IR) was determined by the homeostasis model assessment (HOMA), using the following equation: Insulin resistance (HOMA-IR)=Fasting insulin (μU/mL)×Fasting glucose (mmol/L)/22.5. HOMA-IR has been validated in comparison with the euglycemic/hyperinsulinemic clamp technique in both diabetic and non-diabetic patients.

All patients were tested at the time of biopsy for HCV-RNA (RT-PCR on made; limit of detection: 12 IU/ml). Genotyping was performed by INNO-LiPA, HCV II, Bayer.

sCD36 Plasma Levels

sCD36 was measured on fasting samples by an in-house ELISA as previously described (Handberg A, Levin K, Hojlund K, Beck-Nielsen H. Identification of the oxidized low-density lipoprotein scavenger receptor CD36 in plasma: a novel marker of insulin resistance. Circulation 2006; 114(11): 1169-76). EDTA-plasma was subjected to 3 freeze-thaw cycles before analysis. A pool of EDTA plasma was sonicated 3 times for 5 seconds on ice, applied in 7 dilutions and used as a standard concentration curve. Internal controls were run in quadruplicates on each plate. Runs were accepted if the controls were within ±2 SD from mean, and most were within 1 SD. Intra-assay CV % was 6%, and total day-to-day-assay CV % was 16.4%.

### Histology

Slides were coded and read by one pathologist (D.C.), who was unaware of patients identity and history. A minimum length of 15 mm of biopsy specimen or the presence of at least 10 complete portal tracts was required. Biopsies were classified according to Scheuer numerical scoring system. The percentage of hepatocytes containing macrovescicular fat was determined for each 10× field. An average percentage of steatosis was then determined for the entire specimen. Steatosis was assessed as the percentage of hepatocytes containing fat droplets (minimum 5%), and evaluated as a continuous variable. Steatosis was classified as absent-mild at <20%, or moderate-severe at ≧20%.

### Antiviral Treatment Schedule and Outcomes

Patients were treated with standard antiviral therapy with pegylated interferon (PEG-IFN) α-2a (Pegasys, Roche, Basel, Switzerland) 180 mcg/week plus ribavirin (RBV) at a dosage of 1,000 or 1,200 mg/day according to body weight (1,000 mg/day for a body weight of <75 kg; 1,200 mg/day for a body weight of >75 kg) for 48 weeks. Patients were withdrawn from treatment if they did not achieve a virologic response, defined as undetectable serum HCV RNA by PCR, within 24 weeks after treatment start. This endpoint was in accordance with the stopping rules defined by the European Association for the Study of the Liver (EASL) Consensus Conference on Hepatitis C.

SVR was defined as negative serum HCV RNA on PCR 6 months after stopping anti-viral therapy.

### Statistical Analysis

Continuous variables were summarized as mean±SD, and categorical variables as frequency and percentage. The Student's t-test and chi-square test were used when appropriate. Multiple linear regression analysis was performed to identify independent predictors of sCD36 as the continuous dependent variable. For statistical purposes, the values of sCD36 were entered multiplied by 10 in the regression. As candidate risk factors, we selected age, gender, BMI, WC, the baseline of ALT, γ-glutamil transferase, glucose, insulin, HOMA score, triglycerides, total and HDL cholesterol, platelet count, the presence of diabetes, arterial hypertension, metabolic syndrome, Log10 HCV RNA levels, and finally histological parameters (steatosis, necroinflammatory activity score, and fibrosis).

Multiple logistic regression models were used to assess the relationship of steatosis, and SVR to the demographic, metabolic, and histological characteristics of patients. In the first model, the dependent variable was moderate-severe steatosis, coded as 1=steatosis ≧20% vs. 0=steatosis <20%. In the second model the dependent variable was SVR coded 1=positive vs. 0=negative.

Variables associated with the dependent variable on univariate analysis (probability threshold, p≦0.10) were included in the multivariate regression models. To avoid the effect of colinearity, diabetes, IR, HOMA score, blood glucose levels, and insulin levels, as well as metabolic syndrome and its individual features (WC, hypertension, diabetes, blood glucose, triglycerides and HDL cholesterol) were not included in the same multivariate model. Regression analyses were done using Proc Logistic, Proc Reg and subroutine in SAS (SAS Institute, Inc., Cary, N.C., U.S.A.).

### Results

### Patient Features and Histology

The baseline features of the 175 patients are shown in Table 4. The majority of our patients were in the overweight to obesity range, and nearly a quarter of them were hypertensive. Diabetes was present in 16% of patients, all of them on therapy with oral antidiabetic agents. Mean values for total cholesterol, HDL cholesterol, and triglycerides were within the normal range. Metabolic syndrome was diagnosed in 13% of patients. One patient in three had fibrosis≧3 by Scheuer score, with a high prevalence of moderate/severe necroinflammation (grading 2-3). Half of the cases had histological evidence of steatosis, though of moderate/severe grade in only 44 cases (25%).

TABLE 4
Baseline demographic, laboratory, metabolic and histological
characteristics of 175 patients with genotype 1 Chronic Hepatits C.

| | Genotype 1 Chronic Hepatitis C |
|---|---|
| Variable | (n = 175) |
| Mean Age - yrs | 54.3 ± 11.1 |
| Male Gender | 84 (48) |
| Mean Body Mass Index - Kg/m² | 26.9 ± 4.9 |
| Body Mass Index - Kg/m² | |
| <25 | 63 (36.0) |
| 25-29.9 | 78 (44.5) |
| ≧30 | 34 (19.5) |
| Waist Circumference - cm | 94.3 ± 12.8 |
| Arterial Hypertension | 41 (23.4) |
| Type 2 Diabetes | 29 (16.6) |
| Alanine Aminotransferase - IU/L | 103.6 ± 88.1 |
| Platelet Count - 10³X mmc | 192.6 ± 62.9 |
| γ-glutamil transferase - IU/L | 69.0 ± 74.7 |
| Cholesterol - mg/Dl | 171.7 ± 36.7 |
| HDL Cholesterol - mg/Dl | 55.1 ± 19.1 |
| LDL Cholesterol - mg/Dl | 96.9 ± 32.1 |
| Triglycerides - mg/Dl | 98.8 ± 49.3 |
| Blood Glucose - mg/Dl | 99.1 ± 29.4 |
| Insulin - μU/Ml | 13.4 ± 7.2 |
| HOMA | 3.34 ± 2.22 |
| Metabolic Syndrome | 23 (13.1) |
| sCD36 - arbitrary units | 1.74 ± 1.51 |
| Log HCVRNA | 5.7 ± 0.6 |
| Histology at Biopsy | |
| Steatosis as continuous variable | 13.2 ± 16.6 |
| (percent of total cells) | |
| Steatosis<5% | 88 (50.3) |
| Steatosis≧5% to <20% | 43 (24.6) |
| Steatosis ≧20% | 44 (25.1) |
| Inflammatory activity | |
| 1 | 25 (14.3) |
| 2 | 105 (60.0) |
| 3 | 45 (25.7) |
| Stage of fibrosis | |
| 1 | 33 (18.9) |
| 2 | 80 (46.0) |
| 3 | 31 (17.8) |
| 4 | 30 (17.2) |

Abbreviation:
yrs, years;
IU, international units;
HOMA, homeostasis model assessment;
HDL, high density lipoprotein;
LDL, low density lipoprotein.
Data are given as mean ± standard deviation or as number of case (%).

Factors Associated with sCD36 Plasma Levels

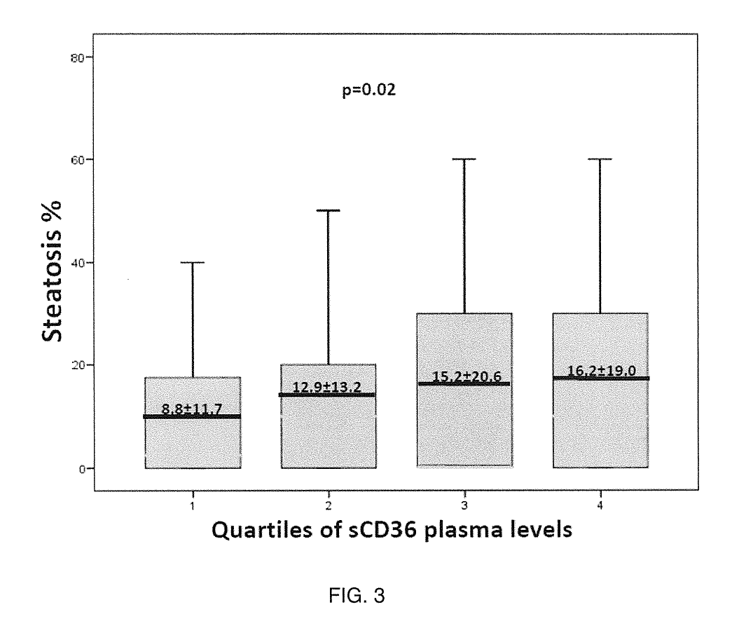

The characteristics of the entire population according to sCD36 quartiles are shown in Table 5. Notably, significantly higher total and LDL cholesterol levels were demonstrated in patients in the first quartile compared to all the others. By contrast, a progressive increase in the severity of steatosis was found from the first to the second, third and finally last quartile (p=0.02) (FIG. 3). Similar data were observed when the analyses were repeated in subjects without diabetes (n=147).

By linear regression analysis only steatosis (p=0.01) was associated with higher sCD36 plasma levels. Similar data were observed in the subset of patients without diabetes.

TABLE 5
Characteristics of 175 patients with genotype 1 Chronic Hepatitis C according to sCD36 quartiles.

| | | | | | P |
|---|---|---|---|---|---|
| | | | | | Value |
| | Quartile 1 | Quartile 2 | Quartile 3 | Quartile 4 | for |
| Characteristic | (No. 48) | (No. 41) | (No. 43) | (No. 43) | trend† |
| CD36 - arbitrary | 0.45 ± 0.19 | 1.02 ± 0.18 | 1.76 ± 0.26 | 3.86 ± 1.52 | — |
| units | | | | | |
| Mean Age - yrs | 53.1 ± 11.6 | 56.1 ± 9.6 | 55.0 ± 11.1 | 53.1 ± 11.8 | 0.95 |
| Male Gender | 25 (52.1) | 14 (34.1) | 14 (32.5) | 21 (48.8) | 0.20 |
| Body Mass Index - | 26.7 ± 5.4 | 28.2 ± 5.0 | 25.9 ± 3.9 | 27.0 ± 5.2 | 0.70 |
| kg/m² | | | | | |
| Waist Circumference - | 96.6 ± 12.0 | 97.6 ± 14.9 | 89.1 ± 10.9 | 93.3 ± 12.3 | 0.15 |
| cm | | | | | |
| AlanineAminotransferase - | 89.9 ± 71.4 | 91.1 ± 70.7 | 111.7 ± 106.1 | 123.0 ± 98.5 | 0.06 |
| IU/L | | | | | |
| γ-glutamyl | 74.3 ± 105.9 | 61.1 ± 45.5 | 60.5 ± 49.4 | 78.9 ± 76.8 | 0.83 |
| transferase - | | | | | |
| IU/L | | | | | |
| Cholesterol - | 187.2 ± 41.5 | 163.8 ± 28.0* | 160.8 ± 33.9* | 172.6 ± 36.1\hat{ } | 0.03 |
| mg/dL | | | | | |
| HDL Cholesterol - | 55.2 ± 21.6 | 55.1 ± 17.5* | 53.4 ± 17.3* | 56.8 ± 19.3* | 0.83 |
| mg/dL | | | | | |
| LDL Cholesterol - | 109.4 ± 33.6 | 90.2 ± 26.3 | 91.2 ± 29.9 | 94.7 ± 34.5 | 0.04 |
| mg/dL | | | | | |
| Triglycerides - | 112.8 ± 63.1 | 91.9 ± 42.6 | 85.7 ± 37.9 | 103.0 ± 44.2 | 0.23 |
| mg/dL | | | | | |
| Platelet Count - | 193.5 ± 66.9 | 188.5 ± 48.5 | 187.5 ± 70.9 | 200.8 ± 63.4 | 0.60 |
| 103X mmc | | | | | |
| Blood Glucose - | 99.5 ± 30.6 | 99.0 ± 30.7 | 100.8 ± 29.5 | 97.1 ± 27.6 | 0.80 |
| mg/dL | | | | | |
| Insulin - μU/Ml | 14.2 ± 7.3 | 13.8 ± 7.1 | 12.8 ± 7.7 | 12.7 ± 6.9 | 0.20 |
| HOMA | 3.56 ± 2.16 | 3.50 ± 2.48 | 3.15 ± 2.28 | 3.11 ± 2.00 | 0.43 |
| Diabetes | 6 | 8 | 10 | 5 | 0.39 |
| Arterial Hypertension | 10 | 7 | 14 | 4 | 0.37 |
| Metabolic Syndrome | 7 | 6 | 4 | 6 | 0.86 |
| Log HCVRNA | 5.8 ± 0.6 | 5.6 ± 0.6 | 5.6 ± 0.7 | 5.6 ± 0.4 | nd |
| Steatosis (continuous | 8.8 ± 11.7 | 12.9 ± 13.2 | 15.2 ± 20.6 | 16.2 ± 19.0 | 0.02 |
| variable) | | | | | |
| Inflammatory | 2.1 ± 0.7 | 2.2 ± 0.7 | 2.0 ± 0.7 | 2.1 ± 0.5 | 0.88 |
| activity | | | | | |
| Stage of fibrosis | 2.2 ± 0.9 | 2.4 ± 0.9 | 2.4 ± 1.0 | 2.2 ± 1.0 | 0.96 |

Abbreviation:
yrs, years;
IU, international units;
HOMA, homeostasis model assessment;
HDL, high density lipoprotein;
LDL, low density lipoprotein.
Data are given as mean ± standard deviation or as number of case.
*p < 0.05 versus quartile 1;
\hat{ }p = 0.07 versus quartile 1.

Factors Associated with Steatosis

High BMI (p=0.04), ALT (p=0.004), γ-glutamyl transferase (p<0.001), triglycerides (p=0.09), insulin (p=0.002), blood glucose (p=0.02), HOMA score (p<0.001), sCD36 (p=0.01), and the presence of the metabolic syndrome (p=0.06), were all associated with steatosis as continuous variable, though only γ-glutamyl transferase (p=0.02), HOMA score (p=0.002), and sCD36 (p=0.04) were maintained as independent factors in multiple linear regression analysis (Table 6). Similar data were obtained after exclusion of diabetic patients.

TABLE 6
Univariate and multivariate analysis of factors associated with steatosis as
continuous variable in 175 patients with genotype 1 Chronic Hepatitis C.

| | Univariate Analysis | | | Multivariate Analysis | | |
|---|---|---|---|---|---|---|
| Variable | β | S.E. | p value | β | S.E. | p value |
| Mean Age - yrs | 0.115 | 0.107 | 0.12 | | — | |
| Male Gender | −0.023 | 2.462 | 0.75 | | — | |
| Mean Body Mass | 0.162 | 0.027 | 0.04 | 0.090 | 0.276 | 0.27 |
| Index - kg/m² | | | | | | |
| Waist | 0.168 | 0.130 | 0.10 | | — | |
| Circumference - cm | | | | | | |
| Alanine | 0.216 | 0.013 | 0.004 | 0.113 | 0.018 | 0.21 |
| Aminotransferase - | | | | | | |
| IU/L | | | | | | |
| γ-glutamyl transferase | 0.272 | 0.016 | <0.001 | 0.201 | 0.020 | 0.02 |
| Cholesterol - mg/dL | 0.042 | 0.033 | 0.56 | | — | |
| HDL | −0.003 | 0.065 | 0.96 | | — | |
| Cholesterol - mg/dL | | | | | | |
| LDL | 0.001 | 0.037 | 0.99 | | — | |
| Cholesterol - mg/dL | | | | | | |
| Triglycerides - mg/dL | 0.128 | 0.025 | 0.09 | 0.010 | 0.030 | 0.90 |
| Platelet Count - | 0.031 | 0.001 | 0.67 | | — | |
| 10³X mmc | | | | | | |
| Blood Glucose - | 0.142 | 0.070 | 0.02 | | — | |
| mg/dL | | | | | | |
| Insulin - μU/Ml | 0.242 | 0.176 | 0.002 | | — | |
| HOMA | 0.283 | 0.561 | <0.001 | 0.270 | 0.620 | 0.002 |
| sCD36 - arbitrary units | 0.179 | 0.822 | 0.01 | 0.163 | 0.964 | 0.04 |
| Diabetes | 0.113 | 3.372 | 0.13 | | — | |
| Arterial Hypertension | 0.109 | 2.962 | 0.15 | | | — |
| Metabolic Syndrome | 0.139 | 3.699 | 0.06 | | | — |
| Log HCVRNA | −0.131 | 2.446 | 0.18 | | | |
| Histology at Biopsy | | | | | | |
| Inflammatory activity | −0.017 | 1.848 | 0.82 | | — | |
| Stage of fibrosis | 0.047 | 1.220 | 0.53 | | — | |

Abbreviation:
β, β coefficient;
S.E., standard error of β;
yrs, years;
IU, international units;
HOMA, homeostasis model assessment;
HDL, high density lipoprotein;
LDL, low density lipoprotein.

The univariate and multivariate comparisons of variables between patients with and without moderate-severe steatosis (≧20%) are reported in Table 7. Again, high BMI, ALT and γ-glutamyl transferase levels, tryglycerides, blood glucose and insulin, HOMA, sCD36, and the presence of the metabolic syndrome were associated with moderate-severe steatosis (p<0.10). In this model, multivariate logistic regression analysis showed that only HOMA (OR 1.243, 95% Cl 1.040-1.484, p=0.01) and sCD36 (OR 1.445, 95% Cl 1.135-1.839, p=0.003) were independently associated with moderate-severe steatosis (≧20%).

TABLE 7
Univariate and multivariate analysis of risk factors associated with steatosis≧20%
in 175 patients with Genotype 1 Chronic Hepatitis C, by logistic regression analysis.

| | | | | Multivariate |
|---|---|---|---|---|
| | | | Univariate | Analysis |
| | Steatosis<20% | Steatosis≧20% | Analysis | OR (95% CI) |
| Variable | n = 131 | n = 44 | p value | p value |
| Age - yrs | 53.9 ± 11.2 | 55.4 ± 10.7 | 0.44 | — |
| Male Gender | 61 | 23 | 0.51 | — |
| Body Mass Index - | 26.6 ± 4.7 | 27.8 ± 5.5 | 0.17 | — |
| Kg/m² | | | | |
| Waist | 93.5 ± 12.9 | 96.6 ± 12.4 | 0.31 | — |
| Circumference - | | | | |
| cm | | | | |
| Alanine | 93.2 ± 82.4 | 135.0 ± 97.7 | 0.007 | 1.004 (1.00-1.008) |
| Aminotransferase - | | | | 0.06 |
| IU/L | | | | |
| Platelet Count - | 193.5 ± 61.5 | 190.2 ± 67.7 | 0.76 | — |
| 10³X mmc | | | | |
| γ-glutamyl | 61.3 ± 71.7 | 91.4 ± 79.6 | 0.02 | 1.002 (0.997-1.007) |
| transferase - | | | | 0.52 |
| IU/L | | | | |
| Cholesterol - | 171.9 ± 36.8 | 171.0 ± 36.9 | 0.89 | — |
| mg/Dl | | | | |
| HDL Cholesterol - | 55.1 ± 18.2 | 55.2 ± 21.9 | 0.98 | — |
| mg/dL | | | | |
| LDL Cholesterol - | 98.2 ± 31.3 | 92.9 ± 34.5 | 0.36 | — |
| mg/dL | | | | |
| Triglycerides - | 94.8 ± 47.0 | 111.2 ± 54.4 | 0.05 | 1.004 (0.996-1.012) |
| mg/dL | | | | 0.34 |
| Blood Glucose - | 96.4 ± 28.7 | 107.1 ± 30.3 | 0.03 | — |
| mg/dL | | | | |
| Insulin - μU/mL | 12.7 ± 6.7 | 15.5 ± 8.3 | 0.03 | — |
| HOMA | 3.07 ± 1.93 | 4.14 ± 2.76 | 0.007 | 1.243 (1.040-1.484) |
| | | | | 0.01 |
| Arterial | 28 | 13 | 0.26 | — |
| Hypertension | | | | |
| Type 2 Diabetes | 18 | 11 | 0.08 | — |
| sCD36 - | 1.53 ± 1.27 | 2.37 ± 1.95 | 0.001 | 1.445 (1.135-1.839) |
| arbitrary units | | | | 0.003 |
| Metabolic | 35 | 9 | 0.10 | — |
| Syndrome | | | | |
| Log HCVRNA | 5.7 ± 0.6 | 5.6 ± 0.6 | 0.32 | |
| Histology at | | | | |
| Biopsy | | | | |
| Inflammatory | 18/79/34 | 7/26/11 | 0.98 | — |
| activity 1/2/3 | | | | |
| Stage of | 23/62/25/20 | 10/18/6/10 | 0.59 | — |
| fibrosis 1/2/3/4 | | | | |

Abbreviation:
y, years;
IU, international units;
HOMA, homeostasis model assessment;
HDL, high density lipoprotein;
LDL, low density lipoprotein.
Data are given as mean ± standard deviation or as number of case (%).

TABLE 8
Univariate analysis of factors associated with sCD36 levels as continuous
variable in 175 patients with Genotype 1 Chronic Hepatitis C.

| | Univariate Analysis | | | |
|---|---|---|---|---|
| Variable | β | S.E. | p value | r value |
| Mean Age - yrs | −0.060 | 0.010 | 0.42 | 0.060 |
| Male gender (n. of cases (%)) | −0.076 | 0.229 | 0.32 | 0.076 |
| Mean Body Mass Index - kg/m² | 0.022 | 0.022 | 0.79 | 0.022 |
| Waist Circumference - cm | −0.103 | 0.013 | 0.32 | 0.103 |
| Alanine Aminotransferase - IU/L | 0.070 | 0.001 | 0.35 | 0.070 |
| γ-glutamyl transferase | −0.020 | 0.002 | 0.79 | 0.020 |
| Cholesterol - mg/dL | −0.045 | 0.003 | 0.55 | 0.045 |
| HDL Cholesterol - mg/dL | 0.039 | 0.006 | 0.61 | 0.039 |
| LDL Cholesterol - mg/dL | −0.069 | 0.004 | 0.37 | 0.069 |
| Triglycerides - mg/dL | −0.041 | 0.002 | 0.59 | 0.041 |
| Platelet Count - 10³X mmc | 0.095 | 0.001 | 0.20 | 0.095 |
| Blood Glucose - mg/dL | −0.080 | 0.004 | 0.29 | 0.080 |
| Insulin - μU/mL | −0.071 | 0.017 | 0.37 | 0.071 |
| HOMA (%) | −0.089 | 0.055 | 0.26 | 0.089 |
| Diabetes | −0.084 | 0.308 | 0.26 | 0.084 |
| Arterial Hypertension | −0.009 | 0.271 | 0.91 | 0.009 |
| Metabolic Syndrome | −0.022 | 0.340 | 0.76 | 0.022 |
| Log HCVRNA | −0.116 | 0.235 | 0.24 | 0.116 |
| Histology at Biopsy | | | | |
| Steatosis | 0.179 | 0.007 | 0.01 | 0.175 |
| Inflammatory activity grade | −0.014 | 0.171 | 0.85 | 0.014 |
| Fibrosis stage | −0.072 | 0.116 | 0.34 | 0.072 |

Abbreviation:
β, β coefficient;
S.E., standard error of β;
y, years;
IU, international units;
HOMA, homeostasis model assessment;
HDL, high density lipoprotein;
LDL, low density lipoprotein.
Note that no factor entered the regression in multivariate analysis after steatosis.

TABLE 9
Univariate and multivariate analysis of factors associated with sustained virological response
in 151 patients with genotype 1 Chronic Hepatitis C by logistic regression analysis.

| | | | | | Multivariate | Partial |
|---|---|---|---|---|---|---|
| | | | Univariate | | Analysis | correlation^{a} |
| | No SVR | SVR | Analysis | r | OR (95% C | R value |
| Variable | n = 84 | n = 67 | p value | value | p value | p value |
| Age - yrs | 54.4 ± 12.0 | 52.9 ± 10.8 | 0.45 | −0.062 | — | |
| Male gender (n. of cases | 35 (41.7) | 36 (53.7) | 0.14 | −0.120 | — | |
| (%)) | | | | | | |
| Body Mass Index - kg/m² | 26.9 ± 4.6 | 26.8 ± 5.1 | 0.87 | −0.014 | — | |
| Waist Circumference - cm | 92.6 ± 11.5 | 95.0 ± 13.0 | 0.36 | −0.097 | — | |
| Alanine | 108.2 ± 96.7 | 97.1 ± 88.9 | 0.47 | −0.059 | — | |
| Aminotransferase - IU/L | | | | | | |
| Platelet Count - 10³X | 193.8 ± 61.5 | 207.3 ± 60.1 | 0.18 | 0.110 | — | |
| mmc | | | | | | |
| γ-glutamyl transferase - | 85.8 ± 93.7 | 51.0 ± 41.5 | 0.006 | −0.224 | 0.992 | −0.165 |
| IU/L | | | | | (0.984-0.999) | 0.04 |
| | | | | | 0.02 | |
| Cholesterol - mg/dL | 170.1 ± 37.0 | 176.2 ± 37.9 | 0.32 | 0.081 | — | |
| HDL Cholesterol - | 56.1 ± 20.9 | 54.1 ± 16.6 | 0.53 | −0.052 | — | |
| mg/dL | | | | | | |
| LDL Cholesterol - mg/dL | 92.8 ± 31.4 | 103.2 ± 35.4 | 0.06 | 0.155 | 1.011 | 0.136 |
| | | | | | (0.999-1.022) | 0.10 |
| | | | | | 0.06 | |
| Triglycerides - mg/dL | 107.8 ± 54.1 | 97.2 ± 44.6 | 0.10 | −0.144 | — | |
| Blood Glucose - mg/dL | 102.0 ± 32.0 | 91.0 ± 19.9 | 0.01 | −0.195 | 0.989 | −0.125 |
| | | | | | (0.974-1.004) | 0.13 |
| | | | | | 0.16 | |
| Insulin - μU/mL | 13.6 ± 6.9 | 13.0 ± 7.7 | 0.64 | −0.040 | — | |
| HOMA (%) | 3.43 ± 2.23 | 3.09 ± 2.21 | 0.37 | −0.076 | — | |
| Arterial Hypertension | 18 (21.4) | 16 (23.9) | 0.72 | 0.029 | — | |
| (%) | | | | | | |
| Type 2 Diabetes (%) | 14 (16.7) | 7 (10.4) | 0.23 | −0.089 | — | |
| sCD36 - arbitrary units | 1.95 ± 1.38 | 1.60 ± 1.82 | 0.19 | −0.108 | — | |
| Metabolic Syndrome (%) | 12 (14.3) | 7 (10.4) | 0.48 | −0.057 | — | |
| Log HCVRNA | 5.7 ± 0.6 | 5.6 ± 0.6 | 0.40 | −0.086 | | |
| Histology at Biopsy | | | | | | |
| Steatosis | 15.2 ± 17.9 | 9.7 ± 14.2 | 0.04 | −0.166 | 0.991 | −0.098 |
| | | | | | (0.968-1.015) | 0.24 |
| Inflammatory activity | 12/49/23 | 10/40/17 | 0.83 | −0.006 | 0.45 | |
| grade (1/2/3) | | | | | | |
| Fibrosis stage (1/2/3/4) | 18/36/12/18 | 10/40/16/1 | 0.002 | −0.128 | −1.017 | −0.037 |
| | | | | | (0.673-1.538) | 0.66 |
| | | | | | 0.93 | |

Abbreviation:
y, years;
IU, international units;
HOMA, homeostasis model assessment;
HDL, high density lipoprotein;
LDL, low density lipoprotein.
Data are given as mean ± standard deviation or as number of case (%).
^{a}After controlling for the other variables significant at univariate analysis.

Factors Associated with SVR

One hundred fifty-one patients underwent and completed the antiviral treatment program. SVR was achieved in 67 patients (44.4%). High γ-glutamyl transferase, low LDL, high blood glucose, steatosis and fibrosis were all associated with lack of SVR at a threshold of p<0.10. By logistic regression, high γ-glutamyl transferase (OR 0.992; 95% Cl 0.984-0.999; p=0.02) was the only independent negative predictor of SVR.

### Discussion

In a cohort of patients with G1 CHC, it is shown that the plasma levels of sCD36 are directly and independently associated with the severity of liver steatosis, together with other well-known metabolic risk factors for fatty liver accumulation.

A growing amount of evidence has shown that liver steatosis, present in about 50% of G1 CHC patients, is a crucial feature in HCV-infected patients, being able to interfere with the natural history of the disease. In this example, we confirmed IR as the key feature in the presence of liver steatosis in patients with G1 CHC, and identified, for the first time, high plasma levels of sCD36 as a factor independently linked to steatosis. In particular, in this setting of patients we found a linear and direct relation between sCD36 and biopsy-proven liver steatosis, also observing a progressive increase of the severity of steatosis according to sCD36 quartiles. The relationship between sCD36 and liver steatosis in G1 CHC patients could also suggest the existence of new molecular mediators and new metabolic pathways potentially involved in the pathogenesis of steatosis.

In conclusion, higher sCD36 plasma levels, together with IR, are independently associated with steatosis and its severity in G1 CHC patients. These data identify a new noninvasive marker of liver steatosis, potentially useful in the follow-up of patients. Thus, from a clinical and diagnostic perspective, the results support the use of sCD36 as an indicator of ectopic liver fat accumulation.

### Sequences

### CD36 Sequences

The CD36 polypeptide and/or CD36 nucleic acid molecule assessed by the method according preferably have one of the sequences shown below. Furthermore, a nucleic acid molecule may be a RNA nucleic acid molecule being complementary to one or more of the sequences shown below:

### Gene Symbol CD36 HGNC

### Chromosomal Location 7q11.2

### UniGene ID

### Build 191 (7 May 2006)

### Hs. 120949 NCBI (FULL LENGTH)

### Hs. 248425

### Hs. 75613

### Hs. 325823

Entrez Gene 948 Entrez gene

### HGNC:1663

### HPRD: 01430

### SwissProt

### P16671 EMBL-EBI

### OMIM

### 173510 NCBI

### 248310 NCBI

### 608404 NCBI

### Reference Sequences

### Protein ID

### NP_{—}000063.2 NCBI

### NP_{—}001001547.1 NCBI

### NP_{—}001001548.1 NCBI

### Transcript ID

### NM_{—}000072 NCBI

### NM_{—}001001547 NCBI

### NM_{—}001001548 NCBI

### Nucleotide Sequence:

| NM_001001548 2338 bp mRNA linear PRI 20 JAN. 2008 | |
|---|---|
| Homo sapiens CD36 molecule (thrombospondin receptor) (CD36), transcript | |
| variant 1, mRNA. | |
| ACCESSION NM_001001548 (VERSION NM_001001548.1) | |
| ORIGIN | |
| (SEQ ID NO.: 1) | |
| 1 | gaggactgca gtgtaggact ttcctgcaga ataccatttg atcctattaa gaattgtcca |
| 61 | aatgttggag catttgattg aaaaatcctt cttagccatt ttaaagatag ctttccaatg |
| 121 | attagacgaa ttgattcttt ctgtgactca tcagttcatt tcctgtaaaa ttcatgtctt |
| 181 | gctgttgatt tgtgaataag aaccagagct tgtagaaacc actttaatca tatccaggag |
| 241 | tttgcaagaa acaggtgctt aacactaatt cacctcctga acaagaaaaa tgggctgtga |
| 301 | ccggaactgt gggctcatcg ctggggctgt cattggtgct gtcctggctg tgtttggagg |
| 361 | tattctaatg ccagttggag acctgcttat ccagaagaca attaaaaagc aagttgtcct |
| 421 | cgaagaaggt acaattgctt ttaaaaattg ggttaaaaca ggcacagaag tttacagaca |
| 481 | gttttggatc tttgatgtgc aaaatccaca ggaagtgatg atgaacagca gcaacattca |
| 541 | agttaagcaa agaggtcctt atacgtacag agttcgtttt ctagccaagg aaaatgtaac |
| 601 | ccaggacgct gaggacaaca cagtctcttt cctgcagccc aatggtgcca tcttcgaacc |
| 661 | ttcactatca gttggaacag aggctgacaa cttcacagtt ctcaatctgg ctgtggcagc |
| 721 | tgcatcccat atctatcaaa atcaatttgt tcaaatgatc ctcaattcac ttattaacaa |
| 781 | gtcaaaatct tctatgttcc aagtcagaac tttgagagaa ctgttatggg gctataggga |
| 841 | tccatttttg agtttggttc cgtaccctgt tactaccaca gttggtctgt tttatcctta |
| 901 | caacaatact gcagatggag tttataaagt tttcaatgga aaagataaca taagtaaagt |
| 961 | tgccataatc gacacatata aaggtaaaag gaatctgtcc tattgggaaa gtcactgcga |
| 1021 | catgattaat ggtacagatg cagcctcatt tccacctttt gttgagaaaa gccaggtatt |
| 1081 | gcagttcttt tcttctgata tttgcaggtc aatctatgct gtatttgaat ccgacgttaa |
| 1141 | tctgaaagga atccctgtgt atagatttgt tcttccatcc aaggcctttg cctctccagt |
| 1201 | tgaaaaccca gacaactatt gtttctgcac agaaaaaatt atctcaaaaa attgtacatc |
| 1261 | atatggtgtg ctagacatca gcaaatgcaa agaagggaga cctgtgtaca tttcacttcc |
| 1321 | tcattttctg tatgcaagtc ctgatgtttc agaacctatt gatggattaa acccaaatga |
| 1381 | agaagaacat aggacatact tggatattga acctataact ggattcactt tacaatttgc |
| 1441 | aaaacggctg caggtcaacc tattggtcaa gccatcagaa aaaattcaag tattaaagaa |
| 1501 | tctgaagagg aactatattg tgcctattct ttggcttaat gagactggga ccattggtga |
| 1561 | tgagaaggca aacatgttca gaagtcaagt aactggaaaa ataaacctcc ttggcctgat |
| 1621 | agaaatgatc ttactcagtg ttggtgtggt gatgtttgtt gcttttatga tttcatattg |
| 1681 | tgcatgcaga tcgaaaacaa taaaataaac ctggctcaag cacaaaccaa tttgtgttgt |
| 1741 | tctgattcaa taattggttt ctgggtggcc aattcagaag aagagtgtac atgctcaaca |
| 1801 | aatcctaggc cctgcattcc tgtcatcctc atccggggga aacaccatca tcccagtagc |
| 1861 | tgccctattc aactgcaaca gtctccagga ccatcagtat actgcatttc atgtgcacca |
| 1921 | aatattttga aagacattta taaataattg gcttatgact catatttctc tatgaatacc |
| 1981 | ttcatacagc aggtataact cttttcttta tgggcttaaa tattttgtca ctgatcctgc |
| 2041 | aaatggacat cattttagca cactagcggt ttatatttta aggaccttca ttctctgttc |
| 2101 | tgcacctctt ctggaaattg agtaaatttt gctttttttt ttttactcag ttgcaactta |
| 2161 | cgcttggcat cttcagaatg cttttctagc attaagagat gtaaatgata aaggaattat |
| 2221 | tgtatgaaat attacaaagc gtagactatg cattgttatt cattataata ttttttgctg |
| 2281 | tcataatcgc ctcataaaga caggtttcaa ccattaaaat atgttcttcc ttaaaaaa |
| // | |
| ACCESSION NM_000072 (VERSION NM_000072.2) | |
| NM_000072 1983 bp mRNA linear PRI 20 JAN. 2008 | |
| DEFINITION Homo sapiens CD36 molecule (thrombospondin receptor) (CD36), | |
| transcript variant 3, mRNA. | |
| ORIGIN | |
| (SEQ ID NO.: 2) | |
| 1 | gaggactgca gtgtaggact ttcctgcaga ataccatttg atcctattaa gaattgtcca |
| 61 | aatgttggag catttgattg aaaaatcctt cttagccatt ttaaagatag ctttccaatg |
| 121 | attagacgaa ttgattcttt ctgtgactca tcagttcatt tcctgtaaaa ttcatgtctt |
| 181 | gctgttgatt tgtgaataag aaccagagct tgtagaaacc actttaatca tatccaggag |
| 241 | tttgcaagaa acaggtgctt aacactaatt cacctcctga acaagaaaaa tgggctgtga |
| 301 | ccggaactgt gggctcatcg ctggggctgt cattggtgct gtcctggctg tgtttggagg |
| 361 | tattctaatg ccagttggag acctgcttat ccagaagaca attaaaaagc aagttgtcct |
| 421 | cgaagaaggt acaattgctt ttaaaaattg ggttaaaaca ggcacagaag tttacagaca |
| 481 | gttttggatc tttgatgtgc aaaatccaca ggaagtgatg atgaacagca gcaacattca |
| 541 | agttaagcaa agaggtcctt atacgtacag agttcgtttt ctagccaagg aaaatgtaac |
| 601 | ccaggacgct gaggacaaca cagtctcttt cctgcagccc aatggtgcca tcttcgaacc |
| 661 | ttcactatca gttggaacag aggctgacaa cttcacagtt ctcaatctgg ctgtggcagc |
| 721 | tgcatcccat atctatcaaa atcaatttgt tcaaatgatc ctcaattcac ttattaacaa |
| 781 | gtcaaaatct tctatgttcc aagtcagaac tttgagagaa ctgttatggg gctataggga |
| 841 | tccatttttg agtttggttc cgtaccctgt tactaccaca gttggtctgt tttatcctta |
| 901 | caacaatact gcagatggag tttataaagt tttcaatgga aaagataaca taagtaaagt |
| 961 | tgccataatc gacacatata aaggtaaaag gaatctgtcc tattgggaaa gtcactgcga |
| 1021 | catgattaat ggtacagatg cagcctcatt tccacctttt gttgagaaaa gccaggtatt |
| 1081 | gcagttcttt tcttctgata tttgcaggtc aatctatgct gtatttgaat ccgacgttaa |
| 1141 | tctgaaagga atccctgtgt atagatttgt tcttccatcc aaggcctttg cctctccagt |
| 1201 | tgaaaaccca gacaactatt gtttctgcac agaaaaaatt atctcaaaaa attgtacatc |
| 1261 | atatggtgtg ctagacatca gcaaatgcaa agaagggaga cctgtgtaca tttcacttcc |
| 1321 | tcattttctg tatgcaagtc ctgatgtttc agaacctatt gatggattaa acccaaatga |
| 1381 | agaagaacat aggacatact tggatattga acctataact ggattcactt tacaatttgc |
| 1441 | aaaacggctg caggtcaacc tattggtcaa gccatcagaa aaaattcaag tattaaagaa |
| 1501 | tctgaagagg aactatattg tgcctattct ttggcttaat gagactggga ccattggtga |
| 1561 | tgagaaggca aacatgttca gaagtcaagt aactggaaaa ataaacctcc ttggcctgat |
| 1621 | agaaatgatc ttactcagtg ttggtgtggt gatgtttgtt gcttttatga tttcatattg |
| 1681 | tgcatgcaga tcgaaaacaa taaaataagt aagtatgtac caaaaaatat tgcttcaata |
| 1741 | atattagctt atatattact tgttttcact ttatcaaaga gaagttacat attaggccat |
| 1801 | atatatttct agacatgtct agccactgat catttttaaa tataggtaaa taaacctata |
| 1861 | aatattatca cgcagatcac taaagtatat ctttaattct gggagaaatg agataaaaga |
| 1921 | tgtacttgtg accattgtaa caatagcaca aataaagcac ttgtgccaaa gttgtccaaa |
| 1981 | aaa |
| ACCESSION NM_001001547 (VERSION NM_001001547.1) | |
| NM_001001547 2050 bp mRNA linear PRI 20 JAN. 2008 | |
| Homo sapiens CD36 molecule (thrombospondin receptor) (CD36), | |
| transcript variant 2, mRNA. | |
| ORIGIN | |
| (SEQ ID NO.: 3) | |
| 1 | agatgtcagg ataaccttaa ggatagatga agggttgaga gcctgtgcct catttctgag |
| 61 | ttctcagctg ctatgccgtg gaaatcctgt ttactttctg catctgctcc tgcaagactc |
| 121 | tggagccagt cttgaggtcc tacatctccg aaagcaagct cttctagaag ttgatagctt |
| 181 | tccaatgatt agacgaattg attctttctg tgactcatca gttcatttcc tgtaaaattc |
| 241 | atgtcttgct gttgatttgt gaataagaac cagagcttgt agaaaccact ttaatcatat |
| 301 | ccaggagttt gcaagaaaca ggtgcttaac actaattcac ctcctgaaca agaaaaatgg |
| 361 | gctgtgaccg gaactgtggg ctcatcgctg gggctgtcat tggtgctgtc ctggctgtgt |
| 421 | ttggaggtat tctaatgcca gttggagacc tgcttatcca gaagacaatt aaaaagcaag |
| 481 | ttgtcctcga agaaggtaca attgctttta aaaattgggt taaaacaggc acagaagttt |
| 541 | acagacagtt ttggatcttt gatgtgcaaa atccacagga agtgatgatg aacagcagca |
| 601 | acattcaagt taagcaaaga ggtccttata cgtacagagt tcgttttcta gccaaggaaa |
| 661 | atgtaaccca ggacgctgag gacaacacag tctctttcct gcagcccaat ggtgccatct |
| 721 | tcgaaccttc actatcagtt ggaacagagg ctgacaactt cacagttctc aatctggctg |
| 781 | tggcagctgc atcccatatc tatcaaaatc aatttgttca aatgatcctc aattcactta |
| 841 | ttaacaagtc aaaatcttct atgttccaag tcagaacttt gagagaactg ttatggggct |
| 901 | atagggatcc atttttgagt ttggttccgt accctgttac taccacagtt ggtctgtttt |
| 961 | atccttacaa caatactgca gatggagttt ataaagtttt caatggaaaa gataacataa |
| 1021 | gtaaagttgc cataatcgac acatataaag gtaaaaggaa tctgtcctat tgggaaagtc |
| 1081 | actgcgacat gattaatggt acagatgcag cctcatttcc accttttgtt gagaaaagcc |
| 1141 | aggtattgca gttcttttct tctgatattt gcaggtcaat ctatgctgta tttgaatccg |
| 1201 | acgttaatct gaaaggaatc cctgtgtata gatttgttct tccatccaag gcctttgcct |
| 1261 | ctccagttga aaacccagac aactattgtt tctgcacaga aaaaattatc tcaaaaaatt |
| 1321 | gtacatcata tggtgtgcta gacatcagca aatgcaaaga agggagacct gtgtacattt |
| 1381 | cacttcctca ttttctgtat gcaagtcctg atgtttcaga acctattgat ggattaaacc |
| 1441 | caaatgaaga agaacatagg acatacttgg atattgaacc tataactgga ttcactttac |
| 1501 | aatttgcaaa acggctgcag gtcaacctat tggtcaagcc atcagaaaaa attcaagtat |
| 1561 | taaagaatct gaagaggaac tatattgtgc ctattctttg gcttaatgag actgggacca |
| 1621 | ttggtgatga gaaggcaaac atgttcagaa gtcaagtaac tggaaaaata aacctccttg |
| 1681 | gcctgataga aatgatctta ctcagtgttg gtgtggtgat gtttgttgct tttatgattt |
| 1741 | catattgtgc atgcagatcg aaaacaataa aataagtaag tatgtaccaa aaaatattgc |
| 1801 | ttcaataata ttagcttata tattacttgt tttcacttta tcaaagagaa gttacatatt |
| 1861 | aggccatata tatttctaga catgtctagc cactgatcat ttttaaatat aggtaaataa |
| 1921 | acctataaat attatcacgc agatcactaa agtatatctt taattctggg agaaatgaga |
| 1981 | taaaagatgt acttgtgacc attgtaacaa tagcacaaat aaagcacttg tgccaaagtt |
| 2041 | gtccaaaaaa |

//
CD36 Protein sequence:
(SEQ ID NO.: 4)
MGCDRNCGLI AGAVIGAVLA VFGGILMPVG DLLIQKTIKK QVVLEEGTIA FKNWVKTGTE
VYRQFWIFDV QNPQEVMMNS SNIQVKQRGP YTYRVRFLAK ENVTQDAEDN TVSFLQPNGA
IFEPSLSVGT EADNFTVLNL AVAAASHIYQ NQFVQMILNS LINKSKSSMF QVRTLRELLW
GYRDPFLSLV PYPVTTTVGL FYPYNNTADG VYKVFNGKDN ISKVAIIDTY KGKRNLSYWE
SHCDMINGTD AASFPPFVEK SQVLQFFSSD ICRSIYAVFE SDVNLKGIPV YRFVLPSKAF
ASPVENPDNY CFCTEKIISK NCTSYGVLDI SKCKEGRPVY ISLPHFLYAS PDVSEPIDGL
NPNEEEHRTY LDIEPITGFT LQFAKRLQVN LLVKPSEKIQ VLKNLKRNYI VPILWLNETG
TIGDEKANMF RSQVTGKINL LGLIEMILLS VGVVMFVAFM ISYCACRSKT IK

### Items

The following first set of items 1-22 are preferred embodiments of the invention.

Item 1. A method for diagnosing steatosis or steato-hepatitis in an individual, said method comprising in a sample from said individual

i) determining the concentration of a CD36 polypeptide or part thereof
ii) correlating said concentration determined in i) to a standard level, and
iii) based on said correlation diagnosing said steatosis or steato-hepatitis.

Item 2. The method according to any of the preceding items, wherein said sample is a body fluid sample.

Item 3. The method according to any of the preceding items, wherein said sample is a cell-free sample.

Item 4. The method according to any of the preceding items, wherein said sample is a plasma sample.

Item 5. The method according to any of the preceding items, wherein CD36 is soluble CD36 (sCD36).

Item 6. The method according to any of the preceding items, wherein a level of soluble CD36 above the average level in a population of human beings is indicative of steatosis or steato-hepatitis.

Item 7. The method according to any of the preceding items, wherein a level of soluble CD36 above the sCD36 level of the third quartile of a population of human beings is indicative of steatosis or steato-hepatitis.

Item 8. The method according to any of the preceding items, wherein an increase of the CD36 concentration of at least 1.15 of the standard level is an indication of steatosis or steato-hepatitis.

Item 9. The method according to any of the preceding items, wherein said steatosis or steato-hepatitis is non-alcoholic steatosis or steato-hepatitis.

Item 10. The method according to any of the preceding items, wherein said individual has hepatitis, such as chronic hepatitis C, for example chronic genotype 1 hepatitis C.

Item 11. The method according to any of the preceding items, wherein said concentration of said CD36 polypeptide or part thereof is determined in

i) an amino acid sequence with SEQ ID NO: 4,
ii) an amino acid sequence having at least 90% sequence identity with a sequence of (i), or with a fragment thereof, and/or
iii) an amino acid sequence complementary to any of the sequences of (i) or (ii).

Item 12. The method according to any of the preceding items, wherein the CD36 polypeptide concentration is determined by

i. providing a sample to be investigated
ii. providing an anti-CD36 antibody,
iii. exposing the sample to the anti-CD36 antibody
iv. removing excess unbound antibody
v. optionally, exposing said CD36-antibody complex to at least a further antibody directed against said CD36-antibody complex, and
vi. detecting and quantifying the amount of said antibody

Item 13. The method according to any of the preceding items, wherein the CD36 polypeptide is determined by,

Item 14. providing a plasma sample to be investigated.

Item 15. providing an anti-CD36 antibody,

i. exposing the sample to be investigated to the anti-CD36 antibody bound to a solid phase,

Item 16. optionally exposing the CD36-antibody complex to a second anti-CD36 antibody, and

Item 17. removing excess unbound antibody

Item 18. detecting and quantifying the amount of the antibody which binds to CD36.

Item 19. The method according to any of the preceding items, wherein said CD36 is detected by an immunological method selected from a solid phase ELISA enzyme immunoassay wherein the sample is contacted with said an anti-CD36 antibody bound to a solid phase.

Item 20. The method of item 14, wherein the solid phase is a microtiter plate.

Item 21. The method of any one of the preceding items, wherein said anti-CD36 antibody is selected from the group consisting of monoclonal and polyclonal CD36 specific antibodies.

Item 22. The method of any one of the preceding items, wherein said anti-CD36 antibody is selected from the group of antibodies consisting of sc5522 (CD36 (N-15), goat IgG (epitope N-terminus (h)), sc9154 (CD36 (H-300), rabbit IgG (epitope 1-300 (h)), and sc7309 (CD36 (SMf), mouse IgM).

The following second set of items 1-25 represents further preferred embodiments of the invention

Item 1. A method for determining steatosis or increased likelihood of having or developing steatosis in an individual, said method comprising

i. obtaining a body fluid sample from said individual
ii. contacting the body fluid sample with an anti-CD36 antibody
iii. determining the concentration of a CD36 polypeptide or part thereof in the body fluid
iv. correlating the concentration of CD36 polypeptide or part thereof to a predetermined standard level, and
v. determining that the individual has steatosis or an increased likelihood of developing steatosis based on a concentration of CD36 polypeptide or part thereof above the predetermined standard level.

Item 2. The method of item 1, wherein said sample is a cell-free sample.

Item 3. The method according to any of the preceding items, wherein said sample is a plasma or serum sample.

Item 4. The method according to any of the preceding items, wherein CD36 polypeptide is soluble CD36 (sCD36).

Item 5. The method according to any of the preceding items, wherein the concentration of CD36 polypeptide or part thereof is determined by

i. providing a sample to be investigated
ii. providing an anti-CD36 antibody,
iii. exposing the sample to the anti-CD36 antibody
iv. removing excess unbound antibody
v. optionally, exposing said CD36-antibody complex to a further antibody directed against said CD36-antibody complex, and
vi. detecting and quantifying the amount of said antibody and/or further antibody.

Item 6. The method according to any of the preceding items, wherein the CD36 polypeptide or part thereof is determined by

Item 7. providing a plasma sample to be investigated.

Item 8. providing an anti-CD36 antibody,

i. exposing the sample to be investigated to the anti-CD36 antibody bound to a solid phase,

Item 9. optionally exposing the CD36-antibody complex to a second anti-CD36 antibody, and

Item 10. removing excess unbound antibody

Item 11. detecting and quantifying the amount of the first and/or second antibody which binds to CD36 polypeptide or part thereof.

Item 12. The method according to any of the preceding items, wherein high molecular complexes (e.g. lipid-protein complexes) in the sample are disrupted prior to the exposure of the sample to anti-CD36 antibody.

Item 13. The method according to any of the preceding items, wherein said sample is frozen and thawed at least once, but more preferred 3-4 times, prior to its exposure to anti-CD36 antibody.

Item 14. The method according to any of the preceding items, wherein said CD36 is detected by an immunological method selected from a solid phase ELISA enzyme immunoassay wherein the sample is contacted with said an anti-CD36 antibody bound to a solid phase.

Item 15. The method of item 7, wherein the solid phase is a microtiter plate.

Item 16. The method of any one of the preceding items, wherein said anti-CD36 antibody is selected from the group consisting of monoclonal and polyclonal CD36 specific antibodies.

Item 17. The method of any one of the preceding items, wherein said anti-CD36 antibody is selected from the group of antibodies consisting of sc5522 (CD36 (N-15), goat IgG (epitope N-terminus (h)), sc9154 (CD36 (H-300), rabbit IgG (epitope 1-300 (h)), and sc7309 (CD36 (SMf), mouse IgM).

Item 18. The method according to any of the preceding items, wherein the predetermined level of soluble CD36 is the average level in a population of human beings.

Item 19. The method according to any of the preceding items, wherein a level of soluble CD36 above the sCD36 level of the third quartile of a population of human beings is indicative of steatosis.

Item 20. The method according to any of the preceding items, wherein the predetermined level of the CD36 polypeptide or part thereof is 1.15 of the standard level of a normal human population.

Item 21. The method according to any of the preceding items, wherein said steatosis is non-alcoholic steatosis.

Item 22. The method according to any of the preceding items, wherein said individual has hepatitis, such as chronic hepatitis C, for example chronic genotype 1 hepatitis C.

Item 23. The method according to any of the preceding items, wherein said concentration of said CD36 polypeptide or part thereof is determined in

i) an amino acid sequence with SEQ ID NO: 4,
ii) an amino acid sequence having at least 90% sequence identity with a sequence of (i), or with a fragment thereof, and/or
iii) an amino acid sequence complementary to any of the sequences of (i) or (ii).

Item 24. A method of treating steatosis in an individual, said method comprising in a sample from said individual

i) determining the concentration of a CD36 polypeptide or part thereof
ii) correlating the concentration to a standard level, and
iii) selecting patients having an elevated level of CD36, and
iv) providing a treatment for steatosis.

Item 25. The method according to item 24, wherein said treatment is selected from the group consisting of weight loss (surgical or medical (e.g. using Orlistat, Rimonabant and/or a Glucagon-like protein-1-receptor agonist)), alcohol restriction, diet changes (preferably towards low-carbohydrate diets), physical exercise, improving metabolic risk factors, providing antiglycemic drug agents/Insulin-Sensitizing Medications such as Thiazolidinediones (e.g. Pioglitazone), Metformin, Antioxidant therapy (e.g. vitamin E), Cytoprotective agents (e.g. Ursodeoxycholic acid (UDCA)), 3-hydroxy-3-methylglutaryl-coenzyme, Ezetimibe, and/or Angiotensin-receptor blockers.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for determining steatosis or increased likelihood of having or developing steatosis in an individual, said method comprising
i. obtaining a body fluid sample from said individual
ii. contacting the body fluid sample with an anti-CD36 antibody
iii. determining the concentration of a CD36 polypeptide or part thereof in the body fluid
iv. correlating the concentration of CD36 polypeptide or part thereof to a predetermined standard level, and
v. determining that the individual has steatosis or an increased likelihood of developing steatosis based on a concentration of CD36 polypeptide or part thereof above the predetermined standard level.

2. The method of claim 1, wherein said sample is a cell-free sample.

3. The method of claim 1, wherein said sample is a plasma or serum sample.

4. The method of claim 1, wherein CD36 polypeptide is soluble CD36 (sCD36).

5. The method of claim 1, wherein the concentration of CD36 polypeptide or part thereof is determined by
i) providing a sample to be investigated
ii) providing an anti-CD36 antibody,
iii) exposing the sample to the anti-CD36 antibody
iv) removing excess unbound antibody
v) optionally, exposing said CD36-antibody complex to a further antibody directed against said CD36-antibody complex, and
vi) detecting and quantifying the amount of said antibody and/or further antibody.

6. The method of claim 1, wherein the CD36 polypeptide or part thereof is determined by
i) providing a plasma sample to be investigated,
ii) providing an anti-CD36 antibody,
vii) exposing the sample to be investigated to the anti-CD36 antibody bound to a solid phase,
iii) optionally exposing the CD36-antibody complex to a second anti-CD36 antibody, and
iv) removing excess unbound antibody
v) detecting and quantifying the amount of the first and/or second antibody which binds to CD36 polypeptide or part thereof.

7. The method of claim 5, wherein high molecular complexes (e.g. lipid-protein complexes) in the sample are disrupted prior to the exposure of the sample to anti-CD36 antibody.

8. The method of claim 7, wherein said sample is frozen and thawed at least once, but more preferred 3-4 times, prior to its exposure to anti-CD36 antibody.

9. The method of claim 1, wherein said CD36 is detected by an immunological method selected from a solid phase ELISA enzyme immunoassay wherein the sample is contacted with said an anti-CD36 antibody bound to a solid phase.

10. The method of claim 7, wherein the solid phase is a microtiter plate.

11. The method of claim 5, wherein said anti-CD36 antibody is selected from the group consisting of monoclonal and polyclonal CD36 specific antibodies.

12. The method of claim 11, wherein said anti-CD36 antibody is selected from the group of antibodies consisting of sc5522 (CD36 (N-15), goat IgG (epitope N-terminus (h)), sc9154 (CD36 (H-300), rabbit IgG (epitope 1-300 (h)), and sc7309 (CD36 (SMf), mouse IgM).

13. The method of claim 1, wherein the predetermined level of soluble CD36 is the average level in a population of human beings.

14. The method of claim 13, wherein a level of soluble CD36 above the sCD36 level of the third quartile of a population of human beings is indicative of steatosis.

15. The method of claim 13, wherein the predetermined level of the CD36 polypeptide or part thereof is 1.15 of the standard level of a normal human population.

16. The method of claim 1, wherein said steatosis is non-alcoholic steatosis.

17. The method according to claim 1, wherein said individual has hepatitis, such as chronic hepatitis C, for example chronic genotype 1 hepatitis C.

18. The method of claim 1, wherein said concentration of said CD36 polypeptide or part thereof is determined in
i) an amino acid sequence with SEQ ID NO: 4,
ii) an amino acid sequence having at least 90% sequence identity with a sequence of (i), or with a fragment thereof, and/or
iii) an amino acid sequence complementary to any of the sequences of (i) or (ii).

19. A method of treating steatosis in an individual, said method comprising in a sample from said individual
i) obtaining a body fluid sample from said individual,
ii) contacting the body fluid sample with an anti-CD36 antibody,
iii) determining the concentration of a CD36 polypeptide or part thereof in the body fluid,
iv) correlating the concentration of CD36 polypeptide or part thereof to a predetermined standard level,
v) determining that the individual has steatosis or an increased likelihood of developing steatosis based on a concentration of CD36 polypeptide or part thereof above the predetermined standard level, and
iv) providing a treatment for steatosis.

20. The method according to claim 19, wherein said treatment is selected from the group consisting of weight loss (surgical or medical (e.g. using Orlistat, Rimonabant and/or a Glucagon-like protein-1-receptor agonist)), alcohol restriction, diet changes (preferably towards low-carbohydrate diets), physical exercise, improving metabolic risk factors, providing antiglycemic drug agents/Insulin-Sensitizing Medications such as Thiazolidinediones (e.g. Pioglitazone), Metformin, Antioxidant therapy (e.g. vitamin E), Cytoprotective agents (e.g. Ursodeoxycholic acid (UDCA)), 3-hydroxy-3-methylglutaryl-coenzyme, Ezetimibe, and/or Angiotensin-receptor blockers.


SEQUENCE LISTING (4 sequences)

SEQ ID NO: 1 - DNA, 2338 bp, Homo sapiens
gaggactgcagtgtaggactttcctgcagaataccatttgatcctattaagaattgtccaaatgttggagcatttgattgaaaaatccttcttagccattttaaagatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataaacctggctcaagcacaaaccaatttgtgttgttctgattcaataattggtttctgggtggccaattcagaagaagagtgtacatgctcaacaaatcctaggccctgcattcctgtcatcctcatccgggggaaacaccatcatcccagtagctgccctattcaactgcaacagtctccaggaccatcagtatactgcatttcatgtgcaccaaatattttgaaagacatttataaataattggcttatgactcatatttctctatgaataccttcatacagcaggtataactcttttctttatgggcttaaatattttgtcactgatcctgcaaatggacatcattttagcacactagcggtttatattttaaggaccttcattctctgttctgcacctcttctggaaattgagtaaattttgcttttttttttttactcagttgcaacttacgcttggcatcttcagaatgcttttctagcattaagagatgtaaatgataaaggaattattgtatgaaatattacaaagcgtagactatgcattgttattcattataatattttttgctgtcataatcgcctcataaagacaggtttcaaccattaaaatatgttcttccttaaaaaa

SEQ ID NO: 2 - DNA, 1983 bp, Homo sapiens
gaggactgcagtgtaggactttcctgcagaataccatttgatcctattaagaattgtccaaatgttggagcatttgattgaaaaatccttcttagccattttaaagatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 3 - DNA, 2050 bp, Homo sapiens
agatgtcaggataaccttaaggatagatgaagggttgagagcctgtgcctcatttctgagttctcagctgctatgccgtggaaatcctgtttactttctgcatctgctcctgcaagactctggagccagtcttgaggtcctacatctccgaaagcaagctcttctagaagttgatagctttccaatgattagacgaattgattctttctgtgactcatcagttcatttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtaagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaataaagcacttgtgccaaagttgtccaaaaaa

SEQ ID NO: 4 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK